(19) United States
(12) Patent Application Publication
(10) Pub. No.: US 2024/0099451 A1
(43) Pub. Date: Mar. 28, 2024

(54) DENTAL COMPOSITION AND RELATED DEVICE AND METHOD

(71) Applicant: ZEST IP HOLDINGS, LLC, Carlsbad, CA (US)

(72) Inventors: Zachary Burkett, San Marcos, CA (US); Yuping Li, Irvine, CA (US); Nicholas Aralis, San Juan Capistrano, CA (US)

(21) Appl. No.: 18/216,369

(22) Filed: Jun. 29, 2023

Related U.S. Application Data
(60) Provisional application No. 63/411,010, filed on Sep. 28, 2022.

(51) Int. Cl. A46B 11/00; A61K 8/81; A61Q 11/00

(52) U.S. Cl. CPC A46B 11/0041; A61K 8/8176; A61Q 11/00; A46B 2200/1066; A61K 2800/30; A61K 2800/54; A61K 2800/87

(57) ABSTRACT

An aspect of the disclosure is related to a cleaning composition and a device to apply a cleaning composition. The device may include a container containing a cleaning composition to clean a surface of a dental appliance in an oral cavity, an applicator portion to apply the cleaning composition to the surface of the dental appliance in the oral cavity and a gap between the dental appliance and the oral cavity, the applicator having a portion to be deformed based on the gap width of the gap to be inserted into the gap to apply the cleaning composition in the gap, and an extruder to extrude the cleaning composition in the container through an outlet to the applicator portion and the portion, wherein the cleaning composition is essentially free of a dental abrasive agent, wherein the dental abrasive agent is a solid particle having a hardness harder than the hardness of the surface of the dental appliance.

### PRIORITY CLAIM

This application claims priority benefit under 35 U.S.C. § 119(e) of U.S. provisional application No. 63/411,010, filed Sep. 28, 2022, which is incorporated by reference herein in its entirety.

### BACKGROUND

Oral hygiene plays a pivotal role in oral health and the prevention of oral disease. Poor oral hygiene can result in the accumulation of plaque.

Dental appliances, such as dentures and implants, are to be worn or located in the mouth to aid various functions or supplement a damaged oral cavity. For example, a dental appliance may be used in aligning crooked teeth or replacing a missing tooth. Like teeth, they can be cleaned regularly to maintain cleanness and good oral health. Cleaning a dental appliance may involve removing the dental appliance from the oral cavity. The cleaning process may involve a process of rinsing or soaking. Proper cleaning and care of the dental appliance can be important for oral health. For example, plaque accumulating on teeth can also accumulate on a dental appliance.

### SUMMARY

This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

An aspect of the disclosure may include a device to apply a cleaning composition to a dental appliance disposed in an oral cavity, which may comprise a container containing a cleaning composition to clean a surface of a dental appliance in an oral cavity, an applicator portion to apply the cleaning composition to the surface of the dental appliance in the oral cavity, an extruder to extrude the cleaning composition in the container to the applicator portion, wherein the cleaning composition comprises a solvent, a water-soluble polyvinylpyrrolidone, a cross-linked polyvinylpyrrolidone that is not water-soluble, and a surfactant, wherein the weight ratio of the water-soluble polyvinylpyrrolidone to the cross-linked polyvinylpyrrolidone in the cleaning composition may be from about 0.005 to about 0.05, and wherein the cleaning composition may be essentially free of a dental abrasive agent, wherein the dental abrasive agent may a solid particle having hardness harder than hardness of the surface of the dental appliance.

In some embodiments, the weight ratio of the water-soluble polyvinylpyrrolidone to the cross-linked polyvinylpyrrolidone may be from about 0.01 and to about 0.025.

In some embodiments, the weight percent of the cross-linked polyvinylpyrrolidone may be about 3 wt. % to about 20 wt. %, with respect to the weight of the cleaning composition.

In some embodiments, the applicator portion may include a dispersing portion to disperse the cleaning composition over the surface of the dental appliance.

In some embodiments, the dispersing portion may be a brush to disperse the cleaning composition.

In some embodiments, the device may include a cleaner portion to clean the surface of the dental appliance.

In some embodiments, the cleaner portion may include a brush tip having bristles.

In some embodiments, an average length of the bristles is about 4 millimeter (mm).

In some embodiments, the device may comprise a handle to move the extruder to extrude the cleaning composition.

An aspect of the disclosure is related to a device to apply a cleaning composition to a dental appliance disposed in an oral cavity, comprising a container containing a cleaning composition to clean a surface of a dental appliance in an oral cavity, an applicator portion to apply the cleaning composition to the surface of the dental appliance in the oral cavity, an extruder to extrude the cleaning composition in the container to the applicator portion, wherein the cleaning composition may comprise a solvent, a water-soluble polyvinylpyrrolidone, a cross-linked polyvinylpyrrolidone that is not water-soluble, and a surfactant, and wherein the weight ratio of the water-soluble polyvinylpyrrolidone to the cross-linked polyvinylpyrrolidone in the cleaning composition may be from about 0.005 to about 0.05.

In some embodiments, the cleaning composition may be essentially free of a dental abrasive agent, wherein the dental abrasive agent is a solid particle having hardness harder than hardness of the surface of the dental appliance.

In some embodiments, the cleaning composition may be essentially free of diamond, quartz, silicon carbide, alumina, silica gels and precipitates, calcium carbonate, titanium dioxide, talc, charcoal, and hydrophobic resinous abrasive materials.

In some embodiments, the weight ratio of the water-soluble polyvinylpyrrolidone to the cross-linked polyvinylpyrrolidone may be from about 0.01 and to about 0.025.

In some embodiments, the weight percent of the cross-linked polyvinylpyrrolidone may be about 3 wt. % to about 20 wt. %, with respect to the weight of the cleaning composition.

In some embodiments, the applicator portion may include a dispersing portion to disperse the cleaning composition over the surface of the dental appliance, and wherein the dispersing portion may be a brush to disperse the cleaning composition.

In some embodiments, the device may comprise a cleaner portion to clean the surface of the dental appliance, wherein the cleaner portion may include a brush tip having bristles, and wherein an average length of the bristles may be about 4 mm.

An aspect of the present disclosure is related to a dental appliance cleaning composition to clean a surface of a dental appliance in an oral cavity. In some embodiments, the dental appliance cleaning composition may comprise a solvent, a water-soluble polyvinylpyrrolidone, a cross-linked polyvinylpyrrolidone that is not water-soluble, and a surfactant, wherein the weight ratio of the water-soluble polyvinylpyrrolidone to the cross-linked polyvinylpyrrolidone in the cleaning composition may be from about 0.005 to about 0.05.

In some embodiments, the cleaning composition may be essentially free of a dental abrasive agent, wherein the dental abrasive agent may be a solid particle having hardness harder than hardness of the surface of the dental appliance.

In some embodiments, the weight ratio of the water-soluble polyvinylpyrrolidone to the cross-linked polyvinylpyrrolidone may be from about 0.01 and to about 0.025.

In some embodiments, the weight percent of the cross-linked polyvinylpyrrolidone may be about 3 wt. % to about 20 wt. %, with respect to the weight of the cleaning composition.

An aspect of the disclosure may include a device to apply a cleaning composition, the device may comprise a container to contain a cleaning composition to clean a dental appliance in an oral cavity, an applicator portion to apply the cleaning composition to the dental appliance, an extruder to extrude the cleaning composition in the container to the applicator portion,

According to an aspect of the disclosure, the cleaning composition may comprise a solvent, a mucosal adhesive, and a surfactant.

According to an aspect of the disclosure, the mucosal adhesive may comprise polyvinylpyrrolidone (PVP).

According to an aspect of the disclosure, the cleaning composition may comprise a first type of PVP as the mucosal adhesive and a second type of PVP different from the first type.

According to an aspect of the disclosure, an amount of the PVP can be from about 3 weight percent (wt. %) with respect to a weight of the cleaning composition to about 21 wt. % with respect to the weight of the cleaning composition.

According to an aspect of the disclosure, the surfactant may include sodium lauryl sulfate.

According to an aspect of the disclosure, the applicator portion may include a dispersing portion.

According to an aspect of the disclosure, the dispersing portion can be a brush.

According to an aspect of the disclosure, the device may further comprise a cleaner portion.

According to an aspect of the disclosure, the cleaner portion may include a brush tip.

According to an aspect of the disclosure, the brush tip bristles up to about 4 mm in length.

According to an aspect of the disclosure, the device may further comprise a handle to move the extruder.

The present disclosure is related to a device to apply a cleaning composition to a dental appliance disposed in an oral cavity, wherein the device may comprise a container containing a cleaning composition to clean a surface of a dental appliance in an oral cavity, an applicator portion to apply the cleaning composition to the surface of the dental appliance in the oral cavity and a gap between the dental appliance and the oral cavity, the applicator having a portion to be deformed based on the gap width of the gap to be inserted into the gap to apply the cleaning composition in the gap, and an extruder to extrude the cleaning composition in the container through an outlet to the applicator portion and the portion, wherein the cleaning composition comprises at least one surfactant and water-soluble Polyvinylpyrrolidone (PVP) at a ratio between the at least one surfactant and the water-soluble PVP of from about 0.01 to about 10, wherein the cleaning composition is essentially free of a dental abrasive agent, wherein the dental abrasive agent is a solid particle having a hardness harder than the hardness of the surface of the dental appliance, wherein the cleaning composition exhibits a viscosity of from about 500 cp to about 10,000 cp, and wherein the cleaning composition exhibits about 10% of the cleaning ability of a tooth paste having a pellicle cleaning rate (PCR) value of about 25%.

In some embodiments, the portion may be to be inserted into the gap by 3 millimeter (mm) or more.

In some embodiments, the gap width of the gap may be about 0.3 mm.

In some embodiments, the cleaning composition further comprises a cross-linked polyvinylpyrrolidone (PVP), wherein the weight ratio of the water-soluble PVP to the cross-linked PVP may be from about 0.01 and to about 0.025.

In some embodiments, the weight percent of the cross-linked polyvinylpyrrolidone may be about 3 wt. % to about 20 wt. %, with respect to the weight of the cleaning composition.

In some embodiments, the applicator portion may include a dispersing portion to disperse the cleaning composition over the surface of the dental appliance, wherein the dispersing portion may include the portion to be to be deformed.

In some embodiments, the dispersing portion may be a brush to disperse the cleaning composition.

In some embodiments, the device further comprises a cleaner portion to clean the surface of the dental appliance, wherein the cleaner portion may include the portion to be to be deformed.

In some embodiments, the cleaner portion may include a brush tip having bristles.

In some embodiments, an average length of the bristles may be about 4 mm.

In some embodiments, a handle to move the extruder to extrude the cleaning composition.

The present disclosure is related to a device to apply a cleaning composition to a dental appliance disposed in an oral cavity, wherein the device may comprise a container containing a cleaning composition to clean a surface of a dental appliance in an oral cavity, an applicator portion to apply the cleaning composition to the surface of the dental appliance in the oral cavity, the applicator having a portion to be deformed based on the gap width of the gap to be inserted into the gap to apply the cleaning composition in the gap, and an extruder to extrude the cleaning composition in the container through an outlet to the applicator portion and the portion, wherein the cleaning composition comprises at least one surfactant and water-soluble Polyvinylpyrrolidone (PVP) at a ratio between the at least one surfactant and the water-soluble PVP of from about 0.01 to about 10, wherein the cleaning composition may be essentially free of diamond, quartz, silicon carbide, alumina, silica gels and precipitates, calcium carbonate, titanium dioxide, talc, charcoal, and hydrophobic resinous abrasive materials, wherein the cleaning composition exhibits a viscosity of from about 500 cp to about 10,000 cp, and wherein the cleaning composition exhibits about 10% of the cleaning ability of a tooth paste having a pellicle cleaning rate (PCR) value of about 25%.

In some embodiments, the portion may be to be inserted into the gap by 3 mm or more.

In some embodiments, the portion may be to be inserted into the gap width of the gap may be about 0.3 mm.

In some embodiments, the cleaning composition further comprises a cross-linked polyvinylpyrrolidone (PVP),
wherein the weight ratio of the water-soluble PVP to the cross-linked PVP may be from about 0.01 and to about 0.025.

In some embodiments, the weight percent of the cross-linked polyvinylpyrrolidone may be about 3 wt. % to about 20 wt. %, with respect to the weight of the cleaning composition.

In some embodiments, the applicator portion may include a dispersing portion to disperse the cleaning composition over the surface of the dental appliance,
wherein the dispersing portion may include the portion to be to be deformed, and
wherein the dispersing portion may include a brush to disperse the cleaning composition.

In some embodiments, the device may further comprise a cleaner portion to clean the surface of the dental appliance, wherein the cleaner portion may include the portion to be to be deformed, wherein the cleaner portion may include a brush tip having bristles, and wherein an average length of the bristles may be about 4 mm.

The present disclosure is related to a dental appliance cleaning composition to clean a surface of a dental appliance in an oral cavity, wherein the dental appliance cleaning composition comprises a solvent, a water-soluble polyvinylpyrrolidone (PVP), and at least one surfactant, wherein the cleaning composition comprises at least one surfactant and the water-soluble PVP at a ratio between the at least one surfactant and the water-soluble PVP of from about 0.01 to about 10, wherein the cleaning composition may be essentially free of a dental abrasive agent, wherein the dental abrasive agent may be a solid particle having a hardness harder than the hardness of the surface of the dental appliance, wherein the cleaning composition exhibits a viscosity of from about 500 cp to about 10,000 cp, and wherein the cleaning composition exhibits about 10% of the cleaning ability of a tooth paste having a pellicle cleaning rate (PCR) value of about 25%.

In some embodiments, the cleaning composition further comprises a cross-linked polyvinylpyrrolidone (PVP), wherein the weight ratio of the water-soluble PVP to the cross-linked PVP may be from about 0.01 and to about 0.025.

### BRIEF DESCRIPTION OF THE FIGURES

In this Application:

FIG. 1 illustrates a device to apply a cleaning composition according to an aspect of the disclosure;

FIG. 2 illustrates a device to apply a cleaning composition according to an aspect of the disclosure; and

FIGS. 3A-3C illustrate a cleaner portion in a device to apply a cleaning composition according to an aspect of the disclosure.

FIG. 4 illustrates a cross-sectional side view of a handle attached to a body including a compartment to reserve the toothpaste according to an aspect of the disclosure.

### DETAILED DESCRIPTION

It is to be understood that the disclosure is not limited to the particular embodiments of the present disclosure described below, as variations of the particular embodiments may be made and still fall within the scope of the appended claims. It is also to be understood that the terminology employed is for the purpose of describing particular embodiments, and is not intended to be limiting.

As used herein, the singular forms “a,” “an” and “the” include plural reference unless the context clearly dictates otherwise. Unless defined otherwise, all technical and scientific terms used herein have the same meaning as commonly understood to one of ordinary skill in the art to which this disclosure belongs.

As used herein, the term “oral cavity” refers to the mouth, which includes the lips, the lining inside the cheeks and lips, the front two thirds of the tongue, the upper and lower gums, the floor of the mouth under the tongue, the bony roof the mouth, and the small area behind the wisdom teeth.

As used herein, the term “abrasive”, “abrasive agent”, “dental abrasive agent” or “dental abrasive” is used herein to refer materials that are used to clean teeth and wears or scratches a surface of a dental appliance due to abrasion or other mechanical interactions. An abrasive, abrasive agent, dental abrasive agent or dental abrasive can be of inorganic minerals such as diamond, quartz, silicon carbide, alumina, silica gels and precipitates, calcium carbonate, titanium dioxide, talc, charcoal, and hydrophobic resinous abrasive materials such as particulate condensation products of urea and formaldehyde.

Oral hygiene plays a pivotal role in oral health and the prevention of oral disease. Poor oral hygiene can result in the accumulation of plaque. Plaque is a diverse biofilm that develops as bacterial preferentially attach to surfaces. In oral cavity, the initial phase of adhesions allows microbes to accumulate in the salivary pellicle that forms on the clean oral surfaces, such as teeth and gingivae. The initial attachment is tenuous and reversible but within minutes the bacteria become irreversibly attached. Accumulation of bacteria results in the formation of extracellular polymers which create a sticky hydrated matrix that is difficult to remove. Over time, the dental plaque develops into a complex structure which provide benefits to the host such as resisting colonization by pathogens. If the dental biofilms are not removed by regularly self-performed oral hygiene, they become dysbiotic as their local conditions favor the emergence of pathogenic species. Bacteria of a given species can be present with a variety of phenotypes from rapid growth to dormant within the same biofilm. Ultimately, an oral environment shift occurs, resulting in tooth caries, gingival inflammation, etc. Therefore, oral hygiene needs to be practiced daily using a toothpaste and mechanical toothbrushing in order to prevent plaque accumulation.

Approximately one-third of Americans older than 65 years of age are fully edentulous and can be benefitted from replacement of a missing tooth. An implant-supported overdenture can provide good retention, stability, function and esthetics and can inhibit bone loss.

In an example, when an implant-supported overdenture is installed or fixed in an oral cavity, it may be installed or fixed to be not removable. In an example, a conventional toothbrush is difficult to reach around abutments that the dental appliance such as dentures attaches and the gaps between the gums and the dental appliance such as dentures. As a result, food residues and bacterial can easily accumulate at the position to form dental plaque, resulting in infection, inflammation, oral pain and mouth ulcers which thereby impairing diet and quality of life.

Commonly, typical cleaning such as typical overdenture cleaning use toothbrush and separate toothpaste tube, as well as water pick. However, a dimension of the toothbrush may not be adequate to access different parts of the dental appliance such as dentures. The abrasiveness of the brush for cleaning teeth may be too harsh for the material of the dental appliance and may damage the dental appliance surface. Moreover, abrasives contained in the toothpaste which are selected for cleaning a tooth surface is too harsh for the material of the dental appliance and may also damage the dental appliance surface. For example, abrasives can prematurely wear the dental appliance surface or scratch the surface. Moreover, while the patient is away from home due to, for example, traveling, working in a different location, etc., having to use toothbrush with separate toothpaste or water pick to clean their dental appliances such as an overdenture can be inconvenient or may not be possible.

There is a need for a cleaning composition free of abrasives added generally for teeth cleaning, especially for dental appliance cleaning such as overdenture cleaning, which can still provide the benefits of a regular dentifrice composition. Specifically, there is a need for an abrasive-free cleaning composition which can provide therapeutic and cosmetic hygiene benefits, including but are not limited to, control of plaque and calculus formulation, removal and prevention of stain, refresh breath, safe use on dental appliances, aesthetic mouth feeling such as fresh sensation or slippery sensory feel.

An aspect of the present disclosure is related to a dentifrice brush assembly including a brush having: a handle for holding a brush and having a compartment to reserve a cleaning composition such as dentifrice-cleaning paste; a head having bristles to brush an overdenture; a channel or a conduit running from the compartment to the head, an outlet, a channel or a conduit to facilitate delivery of the cleaning composition in the compartment to the head and the bristles, a knob coupled to the piston to turn the piston to move the toothpaste toward the head of the toothbrush to dispense the toothpaste from the compartment into the bristles of the toothbrush; and head having at least one hole to discharge the toothpaste from the compartment to the toothbrush. The disclosure is related to a cleaning composition or an oral care composition comprising an orally acceptable carrier and are substantially free of abrasive materials, essentially free of abrasive materials, or free of added abrasive materials for a cleaning purpose. The compositions may be present as a gel, a cream, a paste, or present as aqueous or anhydrous compositions.

An aspect of the disclosure is related to a cleaning composition or a dentifrice.

A dentifrice in form of liquid, fluid, semi-solid, gel, paste, powder, or an equivalent for cleaning teeth contains various cleaning agents, actives and abrasives which are formulated that can be suitable for oral hygiene. The paste form is a common form and generally is filled in laminate tubes. Abrasives may include silica, such as silica gel, hydrated silica or precipitated silica, alumina, insoluble phosphates, calcium carbonate, resinous abrasives. These abrasives are used to physically scrub the surface of the teeth through which the food debris, stains and dental plaque films can be removed.

A dental appliance, such as dentures, dental bridges, aligner and retainers, mouthguards, etc., are intended to be worn in the mouth or in the oral cavity to either replace missing teeth, guide the teeth to the right position, or protect the teeth. It is well known that biofilm deposition and growth on dental appliances causes problems for patients, including oral malodor, stomatitis, gingivitis, periodontal disease, staining and unpleasant taste in the oral cavity. Therefore, these dental appliances may also need to be cleaned regularly to maintain good oral health. For removable dental appliances, they can be taken out of the oral cavity for cleaning via soaking in a cleaning batch with a dental appliance cleanser for some time or being brushed with dentifrices or specially formulated cleansing creams. Dental appliance cleansers may contain harsh chemicals, such as persulfate, perborate, chlorine bleaching agent, hypochlorite, which cannot be used for directly cleaning a dental appliance in an oral cavity. Brushing with a toothpaste containing abrasives may cause the material of the dental appliance to be scratched or worn out which favors the plaque accumulations and soft tissue inflammation. Therefore, the toothpaste containing abrasives is often not recommended for use on dental appliances.

For fixed dental appliances, there is a need for a gentle or less abrasive cleaning composition free of an abrasive or abrasive agent that can still be effectively in cleaning a dental appliance or both the dental appliance and teeth inside the oral cavity.

There is a need for a cleaning composition such as dentifrice composition free or essentially free of abrasives, which can still provide the benefits of a regular dentifrice composition. Specifically, there is a need for an abrasive-free cleaning composition which can provide therapeutic and cosmetic hygiene benefits, including but are not limited to, control of plaque and calculus formulation, removal and prevention of stain, refresh breath, safe use on dental appliances, aesthetic mouth feeling such as fresh sensation or slippery sensory feel.

A cleaning composition free of abrasives has not been available for directly usage in the oral cavity due to the general understanding by those of ordinary skill in dental or oral hygiene arts, that abrasives are required in cleaning compositions in order to remove plaque and stains.

An aspect of the disclosure is related to a dental appliance. A dental appliance, such as a denture, may be worn in the mouth, for example, to replace missing a tooth. Like a tooth, they should be cleaned regularly, for example, to maintain fresh breath and good oral health. For example, a removable dental appliance can be removed from oral cavity and cleaned with relatively harsh cleaning conditions such as a cleaning solution. For example, a nonremovable dental appliance may not be removed from oral cavity for cleaning. Moreover, there can be a benefit of cleaning a removable dental appliance without having to remove it from the oral cavity. Currently, there are unmet needs for cleaning dental appliances. For example, brushing a dental appliance, such as a dental appliance made with acrylic polymers, with a toothpaste containing abrasives may cause a damage to the material of the appliance, such as being scratched or worn down. For example, a spot on the dental appliance can be missed or overlooked during the brushing process. For example, having to remove a dental appliance from the oral cavity may result in inconvenience to a user of the dental appliance or unintended contamination.

An aspect of the disclosure is related to a cleaning composition that cleans a dental appliance while reducing, suppressing or avoiding a damage to the appliance, the appliance surfaces, or oral tissues.

An aspect of the disclosure is related to a cleaning composition that cleans teeth and a dental appliance while reducing, suppressing or avoiding a damage to the appliance, the appliance surfaces, or oral tissues.

An aspect of the disclosure is related to a device to apply a cleaning composition. For example, an aspect of the disclosure is related to a device to apply a cleaning composition and perform cleaning. For example, a device can include a structure enabling reaching occlude or internal surface area of a dental appliance which cannot be reached by ordinary brushing with a conventional toothbrush. For example, a device can be in a variety of forms or shapes, which may include a form of shape that resembles a toothbrush. For example, compared to a toothbrush, the device may have a relatively small brush head and a sharper tip to reach the occlude or internal surface area of the dental appliance. For example, compared to a toothbrush, the device may have a relatively small brush head and a sharper tip to reach the occlude or internal surface area of the dental appliance. For example, the device may have a tip with an opening (e.g., a single opening, a plurality of openings, an array of openings, etc.) through which the cleaning composition will be discharged or applied onto a surface to be cleaned.

An aspect of the disclosure is related to applying a cleaning composition to a portion, surface, or area of a dental device, such as an area, surface, or portion under a fixed dental prosthesis or around supporting implants or abutments.

An aspect of the disclosure is related to a device to apply a cleaning composition to a portion, surface, or area of a dental device, such as an area, surface, or portion under a fixed dental prosthesis or around supporting implants or abutments.

An aspect of the disclosure is related to using a device to apply a cleaning composition to a portion, surface, or area of a dental device, such as an area, surface, or portion under a fixed dental prosthesis or around supporting implants or abutments.

An aspect of the disclosure is related to a device to apply a cleaning composition. For example, a user such as a patient can use a device and apply a cleaning composition to a dental appliance. The user may then manually clean the dental appliance, for example, with a separate brush or by rinsing with water, fluid or other medium.

An aspect of the disclosure is related to a device to apply a cleaning composition.

An aspect of the disclosure is also related to a device to apply a cleaning composition and perform cleaning. For example, a device can include a structure enabling cleaning occlude or internal surface area which cannot be reached by ordinary brushing. For example, a device can be in a variety of forms, which may include a toothbrush-like form, for example, with a softer brush or with smaller brush. For example, the device may have a tip with an opening (e.g., a single opening, a plurality of openings, an array of openings, etc.) through which the cleaning composition will be discharged or applied onto a surface to be cleaned. For example, compared to a toothbrush, the device may have a relatively small brush head and a sharper tip to reach the occlude or internal surface area of the dental appliance.

An aspect of the disclosure is related to an all-in-one device, such as a toothbrush that applies toothpaste product, which can be meant for a user with a different cleaning need, such as a user to clean dental appliance installed in an oral cavity or any consumer, not just one with a prosthetic or implants.

An aspect of the disclosure is related to a device 100 comprising a body 10, a container 12 to contain a cleaning composition 20 as a cleaning agent, an applicator portion 14, and an extruder 16 to extrude the cleaning composition to the applicator portion 14 through a cleaning composition outlet. The device 100 may include a handle 18 to operate or move the extruder 16. The device 100 may include a dispersing portion 141 to disperse the cleaning composition 20 being applied to a portion, an area, or a surface. The device 100 may include a cleaner portion 19 to clean a portion, area, or surface of a dental appliance in the oral cavity.

An aspect of the disclosure is related to a dental appliance cleaning composition 20 with a relatively lower abrasivity. An aspect of the disclosure is related to a dental appliance cleaning composition 20 with a relatively lower abrasivity compared to tooth paste containing abrasives. An aspect of the disclosure is related to a dental appliance cleaning composition 20 without an added abrasive agent.

An aspect of the disclosure is related to a dental appliance cleaning composition 20 having a relatively low viscosity, such as a viscosity lower than a tooth paste.

In an example, when an implant-supported overdenture is installed or fixed in an oral cavity, it may be installed or fixed to be not removable or to be within the oral cavity for a period of time. In an example, a conventional toothbrush is difficult to reach around an abutment that the dental appliance in the oral cavity such as a denture attached or installed and the gap between the gum and the dental appliance. As a result, food residues and bacterial can easily accumulate at the position to form dental plaque, resulting in infection, inflammation, oral pain and mouth ulcers which thereby impairing diet and quality of life. Accordingly, an aspect of the disclosure is related to a device having an applicator portion 14 or a dispersing portion 141 or cleaner portion 19 that is designed to access through the gaps or clean an area around the gaps. An aspect of the disclosure is related to a device having an applicator portion 14, a dispersing portion 141 or cleaner portion 19 that is designed to and access or clean the abutments.

For example, in some embodiments, the applicator portion 14 or the dispersing portion 141 or cleaner portion 19 may have a portion that is flexible, bendable, deformable, compressible, malleable, soft, pliable, squishable, and/or pressable, such that at least some of the portion can be deformed, compressed, squished, or flexed through or into the gaps or around the abutment. In some embodiments, the applicator portion 14 or the dispersing portion 141 or cleaner portion 19 may have a portion that is flexible, such that at least some of the portion can be flexed through or into the gaps or around the abutment. In some embodiments, the applicator portion 14 or the dispersing portion 141 or cleaner portion 19 may have a portion that is bendable, such that at least some of the portion can be bent into the gaps or around the abutment. In some embodiments, the applicator portion 14 or the dispersing portion 141 or cleaner portion 19 may have a portion that is deformable, such that at least some of the portion can be deformed through or into the gaps or around the abutment. In some embodiments, the applicator portion 14 or the dispersing portion 141 or cleaner portion 19 may have a portion that is compressible, such that at least some of the portion can be compressed through or into the gaps or around the abutment. In some embodiments, the applicator portion 14 or the dispersing portion 141 or cleaner portion 19 may have a portion that is malleable, such that at least some of the portion can be deformed, compressed, squished, or flexed through or into the gaps or around the abutment. In some embodiments, the applicator portion 14 or the dispersing portion 141 or cleaner portion 19 may have a portion that is soft, such that at least some of the portion can be deformed, compressed, squished, or flexed through or into the gaps or around the abutment. In some embodiments, the applicator portion 14 or the dispersing portion 141 or cleaner portion 19 may have a portion that is pliable, such that at least some of the portion can be deformed, compressed, squished, or flexed through or into the gaps or around the abutment. In some embodiments, the applicator portion 14 or the dispersing portion 141 or cleaner portion 19 may have a portion that is squishable, such that at least some of the portion can be deformed, compressed, squished, or flexed through or into the gaps or around the abutment. In some embodiments, the applicator portion 14 or the dispersing portion 141 or cleaner portion 19 may have a portion that is pressable, such that at least some of the portion can be deformed, compressed, squished, or flexed through or into the gaps or around the abutment. In some embodiments, the applicator portion 14 or the dispersing portion 141 or cleaner portion 19 may have a portion that has a degree of deformability, such that at least some of the portion can be deformed, compressed, squished, or flexed through or into the gaps or around the abutment. In some embodiments, the applicator portion 14 or the dispersing portion 141 or cleaner portion 19 may have a portion that has a degree of compressibility, such that at least some of the portion can be deformed, compressed, squished, or flexed through or into the gaps or around the abutment. In some embodiments, the applicator portion 14 or the dispersing portion 141 or cleaner portion 19 may have a portion that has a degree of malleability, such that at least some of the portion can be deformed, compressed, squished, or flexed through or into the gaps or around the abutment. In some embodiments, the applicator portion 14 or the dispersing portion 141 or cleaner portion 19 may have a portion that is has a degree of softness, such that at least some of the portion can be deformed, compressed, squished, or flexed through or into the gaps or around the abutment. In some embodiments, the applicator portion 14 or the dispersing portion 141 or cleaner portion 19 may have a portion that is has a degree of pliability, such that at least some of the portion can be deformed, compressed, squished, or flexed through or into the gaps or around the abutment. In some embodiments, the applicator portion 14 or the dispersing portion 141 or cleaner portion 19 may have a portion that has a degree of squishability, such that at least some of the portion can be deformed, compressed, squished, or flexed through or into the gaps or around the abutment. In some embodiments, the applicator portion 14 or the dispersing portion 141 or cleaner portion 19 may have a portion that has a degree of pressability, such that at least some of the portion can be deformed, compressed, squished, or flexed through or into the gaps or around the abutment.

For example, in some embodiments, the applicator portion 14 or the dispersing portion 141 or cleaner portion 19 may have an elongated portion that is flexible, bendable, deformable, compressible, malleable, soft, pliable, squishable, and/or pressable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is flexible, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is bendable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is deformable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is compressible, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is malleable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is soft, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is pliable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is squishable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is pressable, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that has a degree of deformability, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that has a degree of compressibility, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that has a degree of malleability, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is has a degree of softness, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that is has a degree of pliability, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that has a degree of squishability, such that at least some of the elongated portion can be deformed, compressed, squished, or flexed through or into the gaps or around the abutment. In some embodiments, the applicator portion 14 or the dispersing portion 141 or cleaner portion 19 may have an elongated portion that has a degree of pressability, such that at least some of the elongated portion can be deformed, compressed, squished, or flexed through or into the gaps or around the abutment.

For example, in some embodiments, the applicator portion 14 or the dispersing portion 141 or cleaner portion 19 may have a plurality of portions that is flexible, bendable, deformable, compressible, malleable, soft, pliable, squishable, and/or pressable, such that at least some of the portion can be deformed, compressed, squished, or flexed through or into the gaps or around the abutment. In some embodiments, the applicator portion 14 or the dispersing portion 141 or cleaner portion 19 may have a plurality of portions that is flexible, such that at least some of the portion can be deformed, compressed, squished, or flexed through or into the gaps or around the abutment. In some embodiments, the applicator portion 14 or the dispersing portion 141 or cleaner portion 19 may have a plurality of portions that is bendable, such that at least some of the portion can be deformed, compressed, squished, or flexed through or into the gaps or around the abutment. In some embodiments, the applicator portion 14 or the dispersing portion 141 or cleaner portion 19 may have a plurality of portions that is deformable, such that at least some of the portion can be deformed, compressed, squished, or flexed through or into the gaps or around the abutment. In some embodiments, the applicator portion 14 or the dispersing portion 141 or cleaner portion 19 may have a plurality of portions that is compressible, such that at least some of the portion can be deformed, compressed, squished, or flexed through or into the gaps or around the abutment. In some embodiments, the applicator portion 14 or the dispersing portion 141 or cleaner portion 19 may have a plurality of portions that is malleable, such that at least some of the portion can be deformed, compressed, squished, or flexed through or into the gaps or around the abutment. In some embodiments, the applicator portion 14 or the dispersing portion 141 or cleaner portion 19 may have a plurality of portions that is soft, such that at least some of the portion can be deformed, compressed, squished, or flexed through or into the gaps or around the abutment. In some embodiments, the applicator portion 14 or the dispersing portion 141 or cleaner portion 19 may have a plurality of portions that is pliable, such that at least some of the portion can be deformed, compressed, squished, or flexed through or into the gaps or around the abutment. In some embodiments, the applicator portion 14 or the dispersing portion 141 or cleaner portion 19 may have a plurality of portions that is squishable, such that at least some of the portion can be deformed, compressed, squished, or flexed through or into the gaps or around the abutment. In some embodiments, the applicator portion 14 or the dispersing portion 141 or cleaner portion 19 may have a plurality of portions that is pressable, such that at least some of the portion can be deformed, compressed, squished, or flexed through or into the gaps or around the abutment. In some embodiments, the applicator portion 14 or the dispersing portion 141 or cleaner portion 19 may have a plurality of portions that has a degree of deformability, such that at least some of the portion can be deformed, compressed, squished, or flexed through or into the gaps or around the abutment. In some embodiments, the applicator portion 14 or the dispersing portion 141 or cleaner portion 19 may have a plurality of portions that has a degree of compressibility, such that at least some of the portion can be deformed, compressed, squished, or flexed through or into the gaps or around the abutment. In some embodiments, the applicator portion 14 or the dispersing portion 141 or cleaner portion 19 may have a plurality of portions that has a degree of malleability, such that at least some of the portion can be deformed, compressed, squished, or flexed through or into the gaps or around the abutment. In some embodiments, the applicator portion 14 or the dispersing portion 141 or cleaner portion 19 may have a plurality of portions that is has a degree of softness, such that at least some of the portion can be deformed, compressed, squished, or flexed through or into the gaps or around the abutment. In some embodiments, the applicator portion 14 or the dispersing portion 141 or cleaner portion 19 may have a plurality of portions that is has a degree of pliability, such that at least some of the portion can be deformed, compressed, squished, or flexed through or into the gaps or around the abutment. In some embodiments, the applicator portion 14 or the dispersing portion 141 or cleaner portion 19 may have a plurality of portions that has a degree of squishability, such that at least some of the portion can be deformed, compressed, squished, or flexed through or into the gaps or around the abutment. In some embodiments, the applicator portion 14 or the dispersing portion 141 or cleaner portion 19 may have a plurality of portions that has a degree of pressability, such that at least some of the plurality of portions can be deformed, compressed, squished, or flexed through or into the gaps or around the abutment. In some embodiments, the applicator portion 14 or the dispersing portion 141 or cleaner portion 19 may be made of a polymer such as polybutylene terephthalate (PBT), polyethylene terephthalate (PET), nylon, etc., or any combination thereof. In some embodiments, the applicator portion 14 or the dispersing portion 141 or cleaner portion 19 may be flexible. In some embodiments, during a brushing operation, the applicator portion 14 or the dispersing portion 141 or cleaner portion 19 can bent and reach the occlude or internal surface of the dental appliance. In some embodiments, the flexural modulus of elasticity of the applicator portion 14 or the dispersing portion 141 or cleaner portion 19 may be in a range from about 0.5 to about 10 GPa. For example, a flexural modulus of elasticity of the applicator portion 14 or the dispersing portion 141 or cleaner portion 19 is in the range of 2 to 4 GPa.

In some embodiments, the some of the applicator portion 14 or the dispersing portion 141 or cleaner portion 19 may be deformable. In some embodiments, at least a portion of the some of the applicator portion 14 or the dispersing portion 141 or cleaner portion 19 may be deformed based on the gap between the dental appliance and the oral cavity, such that the portion can be inserted into the gap to apply or disperse the cleaning composition 20 in the gap for cleaning the gap. In some embodiments, the portion of the some of the applicator portion 14 or the dispersing portion 141 or cleaner portion 19 can be deformed to fit into a gap width of about 0.03 mm or more, about 0.05 mm or more, about 0.1 mm or more, about 0.2 mm or more, about 0.3 mm or more, about 0.4 mm or more, about 0.5 mm or more, about 0.6 mm or more, about 0.7 mm or more, about 0.8 mm or more, about 0.9 mm or more, or about 1 mm or more. In some embodiments, the portion of the some of the applicator portion 14 or the dispersing portion 141 or cleaner portion 19 can be deformed to fit into a gap width of from about 0.01 and up to about 10 mm. In some embodiments, the portion of the some of the applicator portion 14 or the dispersing portion 141 or cleaner portion 19 can be deformed to fit into a gap width of from about 0.02 and up to about 5 mm. In some embodiments, the portion of the some of the applicator portion 14 or the dispersing portion 141 or cleaner portion 19 can be deformed to fit into a gap width of from about 0.03 and up to about 2 mm. In some embodiments, the portion of the some of the applicator portion 14 or the dispersing portion 141 or cleaner portion 19 can be deformed to fit into a gap width of from about 0.03 and up to about 1 mm. In some embodiments, the portion of the some of the applicator portion 14 or the dispersing portion 141 or cleaner portion 19 can be deformed to fit into a gap width of from about 0.05 and up to about 0.5 mm. In some embodiments, the portion of the some of the applicator portion 14 or the dispersing portion 141 or cleaner portion 19 may have a length sufficient to apply and disperse the cleaning composition 20 to a depth of the gap. In some embodiments, the length may be about 3 mm or more, 4 mm or more, 5 mm or more, 6 mm or more, 7 mm or more, 8 mm or more, 9 mm or more, 10 mm or more, 11 mm or more, 12 mm or more, 13 mm or more, 14 mm or more, 15 mm or more, 20 mm or more, 25 mm or more, or 30 mm or more. For example, the tip portion of the cleaner portion 19 can be deformed to fit into a gap width of about 0.1 mm. For example, the tip portion of the cleaner portion 19 can be deformed to fit into a gap width of about 0.1 mm.

In some embodiments, the portion of the some of the applicator portion 14 or the dispersing portion 141 or cleaner portion 19 can be deformed to fit into a gap width of about 0.03 mm, about 0.05 mm, about 0.1 mm, about 0.2 mm, about 0.3 mm, about 0.4 mm, about 0.5 mm, about 0.6 mm, about 0.7 mm, about 0.8 mm, about 0.9 mm, or about 1 mm.

In some embodiments, an applicator portion 14 may have a cleaning composition 20 outlet through or into which a cleaning composition 20 can be output or supplied to the applicator portion 14 and to the dispersing portion 141 or to the cleaner portion 19.

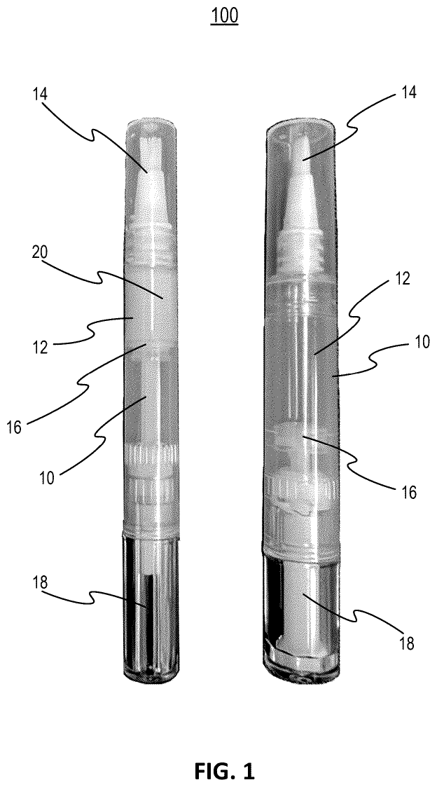

For example, FIG. 1 illustrates a device to apply a cleaning composition 20 according to an aspect of the disclosure. Referring to FIG. 1, the device 100 may include a body 10, a container 12 to contain a cleaning composition 20, an applicator portion 14 that may include a dispersing portion 141, and an extruder 16. The device 100 may include a handle 18. The extruder 16 may be operated or moved by the handle 18.

According to an aspect of the disclosure, the container may have a volume or space to accommodate a cleaning composition 20. The volume of the space may be set based on a variety of factors, such a functional factor or a design factor. For example, the volume of the space may be set based on a portability of the device 100. For example, the volume of the space may be set based on a concentration of the cleaning composition 20 or an amount of cleaning composition 20 to be applied or estimated to be applied. For example, the volume may be up to about 1 milli-Liter (mL). For example, the volume may be up to about 1.5 mL. For example, the volume may be up to about 2.5 mL. For example, the volume may be up to about 3 mL. For example, the volume may be up to about 3.5 mL. For example, the volume may be up to about 4 mL. For example, the volume may be up to about 5 mL. For example, the volume may be up to about 6 mL. For example, the volume may be up to about 7 mL. For example, the volume may be up to about 8 mL. For example, the volume may be up to about 9 mL. For example, the volume may be up to about 10 mL. For example, the volume may be up to about 20 mL. For example, the volume may be up to about 30 mL. For example, the volume may be up to about 40 mL. For example, the volume may be up to about 50 mL. For example, the volume may be up to about 60 mL. For example, the volume may be up to about 70 mL. For example, the volume may be up to about 80 mL. For example, the volume may be up to about 90 mL. For example, the volume may be up to about 100 mL. For example, the volume may be up to about 150 mL. For example, the volume may be up to about 200 mL. For example, the volume may be up to about 250 mL. For example, the volume may be up to about 300 mL. For example, the volume may be up to about 350 mL. For example, the volume may be up to about 400 mL. For example, the volume may be up to about 450 mL. For example, the volume may be up to about 500 mL. For example, the volume may be up to about 600 mL. For example, the volume may be up to about 700 mL. For example, the volume may be up to about 800 mL. For example, the volume may be up to about 900 mL. For example, the volume may be up to about 1 Liter (L). For example, the volume may be up to about 1 L or more. For example, the volume may be from about 1 milli-Liter (mL) to about 1.5 L. For example, the volume may be from about 1.5 milli-Liter (mL) to about 500 mL. For example, the volume may be from about 2 milli-Liter (mL) to about 250 mL. For example, the volume may be from about 1 milli-Liter (mL) to about 100 mL. For example, the volume may be from about 1.5 milli-Liter (mL) to about 50 mL. For example, the volume may be from about 2 milli-Liter (mL) to about 10 mL. For example, the volume may be from about 2.5 milli-Liter (mL) to about 6 mL. For example, the volume may be from about 2.5 mL to about 4 mL. For example, the volume may be about 2.5 mL. For example, the volume may be about 4 mL. For example, the volume may be about 6 mL.

According to an aspect of the disclosure, a device 100 or a body 10 of the device 100 may be in a variety of shapes and sizes, based on various factors, such as a functional or a design factor. For example, the size or the shape of the device 100 or the body 10 may be set based on a portability of the device 100. For example, the size or the shape of the device 100 or the body 10 may be set based on concentration of the cleaning composition 20 or amount of cleaning composition 20 to be applied or estimated to be applied. For example, the device 100 or the body 10 may be a pen style.

According to an aspect of the disclosure, a variety of materials may be used to form a portion of the device 100 or the body 10. For example, the body 10 may be a plastic body such as Poly Propylene, Poly Ethylene, PVC, PET, PETE, etc.

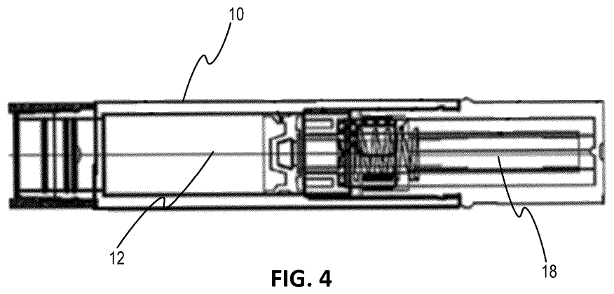

According to an aspect of the disclosure, a cleaning composition 20 may be extruded by an extruder 16. The extruder 16 can be moved by a handle 18. For example, the handle 18 may be a pushable base of the device 100. For example, the handle 18 may be a pullable base of the device 100. For example, the handle 18 may be a twisting base of the device 100. For example, FIG. 4 illustrate a cross-sectional side view of a handle 18 including a twistable base attached to a body 10 that include a compartment to reserve the toothpaste as a container 12 according to an aspect of the disclosure.

According to an aspect of the disclosure, an applicator portion 14 may include a dispersing portion 141. The dispersing portion 141 may be in a variety of shapes and may include a variety of materials to optimize the cleaning. For example, the shape or the material of the dispersing portion 141 may be optimized based on a target cleaning area. In some embodiments, the dispersing portion 141 may be deformable. In some embodiments, at least a portion of the dispersing portion 141 may be deformed based on the gap between the dental appliance and the oral cavity, such that the portion can be inserted into the gap to apply or disperse the cleaning composition 20 in the gap for cleaning the gap. In some embodiments, the portion of the dispersing portion 141 can be deformed to fit into a gap width of about 0.03 mm or more, about 0.05 mm or more, about 0.1 mm or more, about 0.2 mm or more, about 0.3 mm or more, about 0.4 mm or more, about 0.5 mm or more, about 0.6 mm or more, about 0.7 mm or more, about 0.8 mm or more, about 0.9 mm or more, or about 1 mm or more. In some embodiments, the portion of the dispersing portion 141 can be deformed to fit into a gap width of from about 0.01 to about 10 mm. In some embodiments, the portion of the dispersing portion 141 can be deformed to fit into a gap width of from about 0.02 to about 5 mm. In some embodiments, the portion of the dispersing portion 141 can be deformed to fit into a gap width of from about 0.03 to about 2 mm. In some embodiments, the portion of the dispersing portion 141 can be deformed to fit into a gap width of from about 0.03 to about 1 mm. In some embodiments, the portion of the dispersing portion 141 can be deformed to fit into a gap width of from about 0.05 to about 0.5 mm. In some embodiments, the portion of the dispersing portion 141 may have a length sufficient to apply and disperse the cleaning composition 20 to a depth of the gap. In some embodiments, the length may be about 3 mm or more, 4 mm or more, 5 mm or more, 6 mm or more, 7 mm or more, 8 mm or more, 9 mm or more, 10 mm or more, 11 mm or more, 12 mm or more, 13 mm or more, 14 mm or more, 15 mm or more, 20 mm or more, 25 mm or more, or 30 mm or more. For example, the tip portion of the cleaner portion 19 can be deformed to fit into a gap width of about 0.1 mm. For example, the tip portion of the cleaner portion 19 can be deformed to fit into a gap width of about 0.1 mm.

For example, the dispersing portion 141 may be soft bristled with the bristles, for example, similar to a paint brush. For example, the dispersing portion 141 may include a soft brush with bristles or brush hairs. In some embodiments, at least a portion of the soft brush can be deformed based on a gap between the dental appliance and the oral cavity, to be inserted into the gap and to apply and disperse the cleaning composition 20 into the gap. For example, a bristle or brush hair may be deformable, pressable, compressible, or squishable to be inserted into the gap between the dental appliance and the oral cavity. For example, some of the bristles or brush hairs may be deformable, spreadable, or bendable so that at least some of the bristles or brush hairs can be inserted into the gap. For example, some of the bristles or brush hairs may be deformable, spreadable, or bendable and a bristle or brush hair may be deformable, pressable, compressible, or squishable. In some embodiments, some of the bristles or brush hairs may be made of a polymer such as polybutylene terephthalate (PBT), polyethylene terephthalate (PET), nylon, etc., or any combination thereof. In some embodiments, some of the bristles or brush hairs may be flexible. In some embodiments, during a brushing operation, some of the bristles or brush hairs can bent and reach the occlude or internal surface of the dental appliance. In some embodiments, the flexural modulus of elasticity of some of the bristles or brush hairs may be in a range from about 0.5 to about 10 GPa. For example, A flexural modulus of elasticity of some of the bristles or brush hairs is in the range of 2 to 4 GPa. According to an aspect of the disclosure, the applicator portion 14 may include a hollow portion as an outlet so that a cleaning composition 20 may move through the hollow portion in the applicator portion 14.

According to an aspect of the disclosure, a user may use a device 100 to apply a cleaning composition 20 in a variety of situations, for example, to provide accessibility or convenience, such as at home or on the go. The cleaning process can be followed by additional brushing with a different brush or by rinsing.

An aspect of the disclosure is related to a device 100 comprising a body 10, a container 12 to contain a cleaning composition 20, an applicator portion 14, and an extruder 16 to extrude the cleaning composition 20 to the applicator portion 14. The device 100 may include a handle 18 to operate or move the extruder 16. The device 100 may include a dispersing portion 141 to disperse the cleaning composition 20 being applied to a portion, an area, or a surface. The device 100 may include a cleaner portion 19 to clean a portion, area, or surface of a dental appliance in the oral cavity, such as a cleaning brush portion.

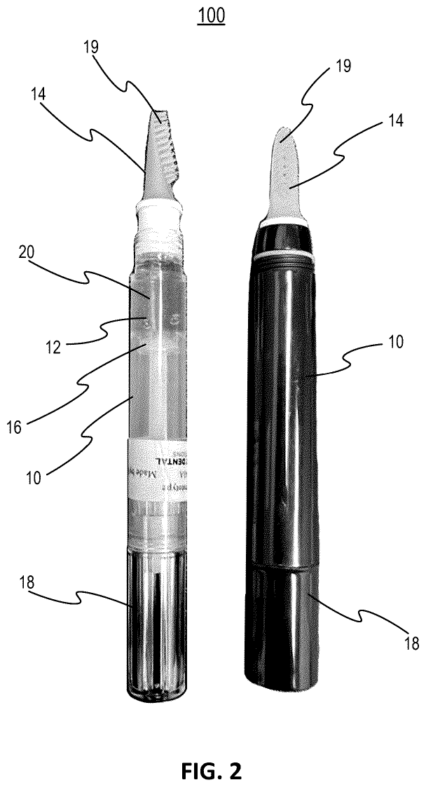

For example, FIG. 2 illustrates a device to apply a cleaning composition 20 according to an aspect of the disclosure. Referring to FIG. 2, the device 100 may include a body 10, a container 12 to contain a cleaning composition 20, an applicator portion 14 that may optionally include a dispersing portion 141, an extruder 16, and a cleaner portion 19 to clean a portion, area, or surface of a dental appliance in an oral cavity. For example, the cleaner portion 19 may include a cleaning brush portion. The extruder 16 may be operated or moved by a handle 18.

According to an aspect of the disclosure, the container 12 may have a volume or space to accommodate a cleaning composition 20. The volume of the space may be set based on a variety of factors, such a function factor or a design factor. For example, the volume of the space may be set based on a portability of the device 100 or based on a concentration of the cleaning composition 20 or an amount of cleaning composition 20 to be or estimated to be applied. For example, the volume may be up to about 1 milli-Liter (mL). For example, the volume may be up to about 1.5 mL. For example, the volume may be up to about 2.5 mL. For example, the volume may be up to about 3 mL. For example, the volume may be up to about 3.5 mL. For example, the volume may be up to about 4 mL. For example, the volume may be up to about 5 mL. For example, the volume may be up to about 6 mL. For example, the volume may be up to about 7 mL. For example, the volume may be up to about 8 mL. For example, the volume may be up to about 9 mL. For example, the volume may be up to about 10 mL. For example, the volume may be up to about 20 mL. For example, the volume may be up to about 30 mL. For example, the volume may be up to about 40 mL. For example, the volume may be up to about 50 mL. For example, the volume may be up to about 60 mL. For example, the volume may be up to about 70 mL. For example, the volume may be up to about 80 mL. For example, the volume may be up to about 90 mL. For example, the volume may be up to about 100 mL. For example, the volume may be up to about 150 mL. For example, the volume may be up to about 200 mL. For example, the volume may be up to about 250 mL. For example, the volume may be up to about 300 mL. For example, the volume may be up to about 350 mL. For example, the volume may be up to about 400 mL. For example, the volume may be up to about 450 mL. For example, the volume may be up to about 500 mL. For example, the volume may be up to about 600 mL. For example, the volume may be up to about 700 mL. For example, the volume may be up to about 800 mL. For example, the volume may be up to about 900 mL. For example, the volume may be up to about 1 Liter (L). For example, the volume may be up to about 1 L or more. For example, the volume may be from about 1 milli-Liter (mL) to about 1.5 L. For example, the volume may be from about 1.5 milli-Liter (mL) to about 500 mL. For example, the volume may be from about 2 milli-Liter (mL) to about 250 mL. For example, the volume may be from about 1 milli-Liter (mL) to about 100 mL. For example, the volume may be from about 1.5 milli-Liter (mL) to about 50 mL. For example, the volume may be from about 2 milli-Liter (mL) to about 10 mL. For example, the volume may be from about 2.5 mL to about 6 mL. For example, the volume may be from about 2.5 mL to about 4 mL. For example, the volume may be about 2.5 mL. For example, the volume may be about 4 mL. For example, the volume may be about 6 mL.

According to an aspect of the disclosure, a device 100 or a body 10 of the device 100 may be in a variety of shapes and sizes, based on one of various factors, such a functional factor or a design factor. For example, the size or the shape of the device 100 or the body 10 may be set based on a portability of the device 100. For example, the size or the shape of the device 100 or the body 10 may be set based on a concentration of the cleaning composition 20 or an amount of the cleaning composition 20 to be or estimated to be applied. For example, the device 100 or the body 10 may be a pen style.

According to an aspect of the disclosure, a variety of materials may be used to form a portion of the device 100 or the body. For example, the body 10 may be a plastic body such as Poly Propylene, Poly Ethylene, PVC, PET, PETE, etc.

According to an aspect of the disclosure, a cleaning composition 20 may be extruded by an extruder 16 moved by as handle 18. For example, the handle 18 may be a pushable base of the device 100. For example, the handle 18 may be a pullable base of the device 100. For example, the handle 18 may be a twisting base.

According to an aspect of the disclosure, an applicator portion 14 may be coupled to a cleaner portion 19, which may be a cleaning brush.

According to an aspect of the disclosure, an applicator portion 14 may include a cleaner portion 19. The cleaner portion 19 may be in a variety of shapes and may include a variety of materials to optimize the cleaning. For example, the shape or the material of the cleaner portion 19 may be optimized based on a target cleaning area. In some embodiments, the cleaner portion 19 may be deformable. In some embodiments, at least a portion of the cleaner portion 19 may be deformed based on the gap between the dental appliance and the oral cavity, such that the portion can be inserted into the gap to apply or disperse the cleaning composition 20 in the gap for cleaning the gap. In some embodiments, the portion of the cleaner portion 19 can be deformed to fit into a gap width of about 0.03 mm or more, about 0.05 mm or more, about 0.1 mm or more, about 0.2 mm or more, about 0.3 mm or more, about 0.4 mm or more, about 0.5 mm or more, about 0.6 mm or more, about 0.7 mm or more, about 0.8 mm or more, about 0.9 mm or more, or about 1 mm or more. In some embodiments, the portion of the cleaner portion 19 can be deformed to fit into a gap width of from about 0.01 to about 10 mm. In some embodiments, the portion of the cleaner portion 19 can be deformed to fit into a gap width of from about 0.02 to about 5 mm. In some embodiments, the portion of the cleaner portion 19 can be deformed to fit into a gap width of from about 0.03 to about 2 mm. In some embodiments, the portion of the cleaner portion 19 can be deformed to fit into a gap width of from about 0.03 to about 1 mm. In some embodiments, the portion of the cleaner portion 19 can be deformed to fit into a gap width of from about 0.05 to about 0.5 mm. In some embodiments, the portion of the cleaner portion 19 may have a length sufficient to apply and disperse the cleaning composition 20 to a depth of the gap. In some embodiments, the length may be about 3 mm or more, 4 mm or more, 5 mm or more, 6 mm or more, 7 mm or more, 8 mm or more, 9 mm or more, 10 mm or more, 11 mm or more, 12 mm or more, 13 mm or more, 14 mm or more, 15 mm or more, 20 mm or more, 25 mm or more, or 30 mm or more. For example, the tip portion of the cleaner portion 19 can be deformed to fit into a gap width of about 0.1 mm.

For example, the cleaner portion 19 may have a tip portion that is narrow enough to be inserted into the gap. For example, the tip portion may be deformable, e.g., pressable, compressible, squishable, etc., to be inserted into the gap. For example, the tip portion of the cleaner portion 19 can be deformed to fit into a gap width of about 0.03 mm or more, about 0.05 mm or more, about 0.1 mm or more, about 0.2 mm or more, about 0.3 mm or more, about 0.4 mm or more, about 0.5 mm or more, about 0.6 mm or more, about 0.7 mm or more, about 0.8 mm or more, about 0.9 mm or more, or about 1 mm or more. In some embodiments, the tip portion of the cleaner portion 19 can be deformed to fit into a gap width of from about 0.01 to about 10 mm. In some embodiments, the tip portion of the cleaner portion 19 can be deformed to fit into a gap width of from about 0.02 to about 5 mm. In some embodiments, the tip portion of the cleaner portion 19 can be deformed to fit into a gap width of from about 0.03 to about 2 mm. In some embodiments, the tip portion of the cleaner portion 19 can be deformed to fit into a gap width of from about 0.03 to about 1 mm. In some embodiments, the tip portion of the cleaner portion 19 can be deformed to fit into a gap width of from about 0.05 to about 0.5 mm. For example, the tip portion of the cleaner portion 19 can be deformed to fit into a gap width of about 0.1 mm.

For example, a brush tip of bristles 191 of the cleaning brush as a cleaner portion 19 may be a firm but flexible plastic brush head. For example, the cleaner portion 19 may be soft bristled with the bristles, for example, similar to a paint brush. For example, the cleaner portion 19 may include a soft brush with bristles or brush hairs. In some embodiments, at least a portion of the soft brush can be deformed based on a gap between the dental appliance and the oral cavity, to be inserted into the gap and to apply and disperse the cleaning composition 20 into the gap. For example, a bristle or brush hair may be deformable, pressable, compressible, or squishable to be inserted into the gap between the dental appliance and the oral cavity. For example, some of the bristles or brush hairs may be deformable, spreadable, or bendable so that at least some of the bristles or brush hairs can be inserted into the gap. For example, some of the bristles or brush hairs may be deformable, spreadable, or bendable and a bristle or brush hair may be deformable, pressable, compressible, or squishable. According to an aspect of the disclosure, the applicator portion 14 may include a hollow portion as an outlet so that a cleaning composition 20 may move through the hollow portion in the applicator portion 14 and extruded out through an outlet 1411 or plurality of outlets 1411.

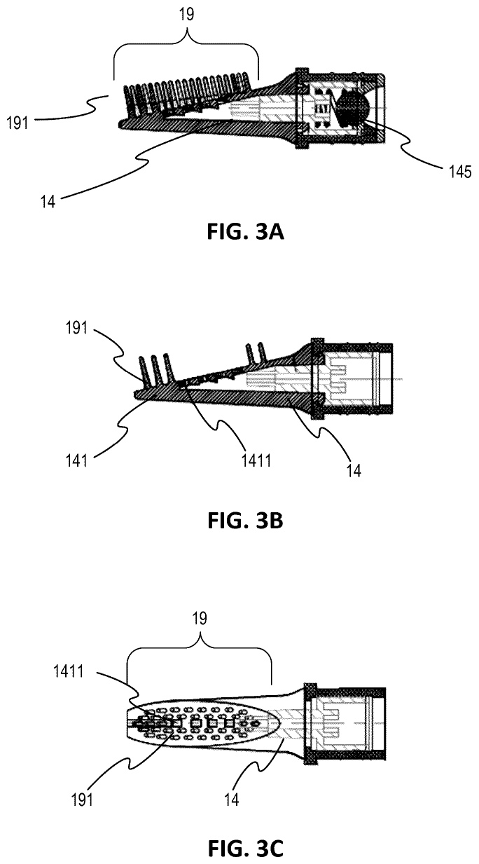

For example, FIGS. 3A-3C illustrate a cleaner portion according to an aspect of the disclosure. Referring to FIGS. 3A-3C, the tip portion of the cleaner portion 19 can be narrow enough to be inserted into the gap between the dental appliance and the oral cavity. For example, the tip portion of the cleaner portion 19 can be deformed to fit into a gap width of about 0.03 mm or more, about 0.05 mm or more, about 0.1 mm or more, about 0.2 mm or more, about 0.3 mm or more, about 0.4 mm or more, about 0.5 mm or more, about 0.6 mm or more, about 0.7 mm or more, about 0.8 mm or more, about 0.9 mm or more, or about 1 mm or more. In some embodiments, the tip portion of the cleaner portion 19 can be deformed to fit into a gap width of from about 0.01 to about 10 mm. In some embodiments, the tip portion of the cleaner portion 19 can be deformed to fit into a gap width of from about 0.02 to about 5 mm. In some embodiments, the tip portion of the cleaner portion 19 can be deformed to fit into a gap width of from about 0.03 to about 2 mm. In some embodiments, the tip portion of the cleaner portion 19 can be deformed to fit into a gap width of from about 0.03 to about 1 mm. In some embodiments, the tip portion of the cleaner portion 19 can be deformed to fit into a gap width of from about 0.05 to about 0.5 mm. For example, the tip portion of the cleaner portion 19 can be deformed to fit into a gap width of about 0.1 mm. In some embodiments, a valve 145, such as a spring and a ball shown in FIG. 3A, may be provided to close the outlet when the cleaning composition is not being extruded.

In some embodiments, some of the tip portion of the cleaner portion 19 may be made of a polymer such as polybutylene terephthalate (PBT), polyethylene terephthalate (PET), nylon, etc., or any combination thereof. In some embodiments, some of the tip portion of the cleaner portion 19 may be flexible. In some embodiments, during a brushing operation, some of the tip portion of the cleaner portion 19 can bent and reach the occlude or internal surface of the dental appliance. In some embodiments, the flexural modulus of elasticity of some of the tip portion of the cleaner portion 19 may be in a range from about 0.5 to about 10 GPa. For example, A flexural modulus of elasticity of some of the tip portion of the cleaner portion 19 is in the range of 2 to 4 GPa.

In some embodiments, the brush tip of bristles 191 may bristles up to various length (e.g., up to about 1 mm in length, up to about 2 mm in length, up to about 3 mm in length, up to about 4 mm in length, up to about 5 mm in length, up to about 6 mm in length, up to about 7 mm in length, up to about 8 mm in length, up to about 9 mm in length, up to about 10 mm in length, or up to about 20 mm in length, 10 mm or more, 11 mm or more, 12 mm or more, 13 mm or more, 14 mm or more, 15 mm or more, 20 mm or more, 25 mm or more, or 30 mm or more in length), to get under part of the dental appliance (e.g., prosthetic and around implants) and to be inserted into the gap between the dental appliance and the oral cavity. Referring to FIGS. 3A-3C, the brush tip of bristles 191 may bristles up to about 4 mm in length, for example, to get under part of prosthetic and around implants. At least a portion of the bristles 191 can be deformed based on a gap between the dental appliance and the oral cavity, to be inserted into the gap and to apply and disperse the cleaning composition 20 into the gap. For example, a bristle 191 may be deformable, pressable, compressible, or squishable to be inserted into the gap between the dental appliance and the oral cavity. For example, some of the bristles 191 may be deformable, spreadable, or bendable so that at least some of the bristles 191 can be inserted into the gap. For example, some of the bristles or brush hairs may be deformable, spreadable, or bendable and a bristle 191 may be deformable, pressable, compressible, or squishable. In some embodiments, at least a portion of the bristles 191 may be made of a polymer such as polybutylene terephthalate (PBT), polyethylene terephthalate (PET), nylon, etc., or any combination thereof. In some embodiments, at least a portion of the bristles 191 may be flexible. In some embodiments, during a brushing operation, at least a portion of the bristles 191 can bent and reach the occlude or internal surface of the dental appliance. In some embodiments, the flexural modulus of elasticity of at least a portion of the bristles 191 may be in a range from about 0.5 to about 10 GPa. For example, A flexural modulus of elasticity of at least a portion of the bristles 191 is in the range of 2 to 4 GPa.

Referring to FIGS. 3A-3C, a plurality of outlets 1411 can be positioned to supply the cleaning composition 20 extruded out of the container 12 around the deformable tip and deformable bristle portion, to apply the cleaning composition 20 in the gap.

According to an aspect of the disclosure, a user may use a device 100 to apply a cleaning composition 20 in a variety of situations to provide, for example, accessibility or convenience, such as at home or on the go. The cleaning process can be performed by a device 100 using a cleaner portion 19, which can then optionally be followed by additional brushing with a different brush or by rinsing.

According to an aspect of the disclosure, a cleaning composition 20 can include a combination of ingredients to increase the cleaning ability while reducing, suppressing or avoiding a damage to a dental appliance. According to an aspect of the disclosure, a cleaning composition 20 can be formulated to reduce or avoid a damage to a dental appliance by abrasion. For example, the cleaning composition 20 may not include an abrasive agent to cause from brushing or from an abrasive agent.

According to an aspect of the disclosure, the cleaning composition 20 may include a solvent. Various materials or combinations of various materials can be used as a solvent. For example, water, various types of alcohol, various types of oils, other types of fluids, or any combination thereof can be used as a solvent.

According to an aspect of the disclosure, the cleaning composition 20 may include a humectant. In some embodiments, a humectant is used to retain moisture in a cleaning composition 20. Various materials or combinations of various materials can be added as a humectant. For example, a humectant can be an agent to bind and hold the solvent in the cleaning composition 20. Examples of humectant includes glycerin, sorbitol, other types of materials, or any combination thereof.

According to an aspect of the disclosure, the cleaning composition 20 may include a sweetener. Various materials or combinations of various materials can be used as a sweetener. For example, the sweetener may include a sugar-less agent, a sugar-free agent, an artificial sweetener, or any combination thereof, such as sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, stevia and cyclamate salts, especially sodium saccharin, stevia, and the mixture thereof.

According to an aspect of the disclosure, the cleaning composition 20 may include a thickener such as an ion-independent thickener. Various materials or combinations of various materials can be used as a thickener. For example, a thickener may include Xanthan gum, hydroxyethyl cellulose, or a combination thereof.

According to an aspect of the disclosure, the cleaning composition 20 may include a flavor carrier. Various materials or combinations of various materials can be used as a flavor carrier.

According to an aspect of the disclosure, the cleaning composition 20 may include a mucosal adhesive. In some embodiments, a mucosal adhesive may enable a bioadhesion to the oral cavity. Various materials or combinations of various materials can be used as a mucosal adhesive.

In some embodiments, various materials or combinations of various materials can be used as a lightweight abrasive or rubbing agent, which may include sodium carboxymethyl cellulose (CMC), hydroxyethyl cellulose (HEC), chitosan, polyvinyl pyrrolidone (PVP) such as water-soluble PVP, and cross-linked polyacrylic acids, other materials types, or any combination thereof.

According to an aspect of the disclosure, the cleaning composition 20 may include a stain remover. Various materials or combinations of various materials can be used as a stain remover.

According to an aspect of the disclosure, the cleaning composition 20 may include a cationic surfactant. Various materials or combinations of various materials can be used as a cationic surfactant. For example, sodium lauryl sulfate can be used as a cationic surfactant.

According to an aspect of the disclosure, the cleaning composition 20 may include an amphiphilic surfactant. Various materials or combinations of various materials can be used as an amphiphilic surfactant. For example, cocamidopropyl betaine can be used as an amphiphilic surfactant.

According to an aspect of the disclosure, the cleaning composition 20 may include a non-ionic surfactant. Various materials or combinations of various materials can be used as a non-ionic surfactant. For example, poloxamer can be used as a non-ionic surfactant.

According to an aspect of the disclosure, the cleaning composition 20 may include a calcium chelator. Various materials or combinations of various materials can be used as a calcium chelator amphiphilic surfactant.

According to an aspect of the disclosure, the cleaning composition 20 may include a plaque disruptor. Various materials or combinations of various materials can be used as a plaque disruptor.

According to an aspect of the disclosure, the cleaning composition 20 may include a pH adjuster or a buffer. Various materials or combinations of various materials can be used as a pH adjuster or a buffer.

According to an aspect of the disclosure, the cleaning composition 20 may include a preservative. Various materials or combinations of various materials can be used as a pH adjuster or a preservative.

According to an aspect of the disclosure, the cleaning composition 20 may include a flavoring agent such as sweet mint flavor, mint flavor, or cool mint favor.

According to an aspect of the disclosure, the cleaning composition 20 may include a coloring agent.

According to an aspect of the disclosure, the cleaning composition 20 may include an antimicrobial agent. Various materials or combinations of various materials can be used as a pH adjuster or an antimicrobial agent.

According to an aspect of the disclosure, the cleaning composition 20 may include an anti-inflammatory agent. Various materials or combinations of various materials can be used as an anti-inflammatory agent.

According to an aspect of the disclosure, the cleaning composition 20 may include an antiplaque agent. Various materials or combinations of various materials can be used as an antiplaque agent.

According to an aspect of the disclosure, the cleaning composition 20 may include an antigingivitis agent. Various materials or combinations of various materials can be used as an antigingivitis agent.

An aspect of the disclosure is related to a cleaning composition or an oral care composition comprising an orally acceptable carrier and are substantially free of an abrasive such as solid particles. The compositions may be present as a gel, a cream, a paste, or present as aqueous or anhydrous compositions.

An aspect of the disclosure is related to a cleaning composition or an oral care composition comprising an orally acceptable carrier, thickeners, surfactants, flavors, and sweeteners.

An aspect of the disclosure is related to a cleaning composition or an oral care composition comprising, with respect to the weight of the composition, a humectant (from about 10 weight percent (wt. %) to about 60 wt. %), a thickening agent (from about 0.1 wt. % to about 25 wt. %), a surfactant (from about 0.2 wt. % to about 10 wt. %), an essential oil component (from about 0.01 wt. % to about 3 wt. %) a coloring agent (from about 0.01 wt. % to about 0.5 wt. %) and water (from about 2 wt. % to about 40 wt. %). It contains 1-20 wt. % hydrophilic polymeric particulates having mean particle size in the range of 3 to 10 μm, with at least 90 wt. % of the particles having a size below 15 μm.

An aspect of the disclosure is related to a brush device comprising: a cleaning composition container; a head mounted to the container, the head comprising an outer surface and a plurality of tooth cleaning elements extending outwardly from the outer surface, and an oral care composition twisted out from the container to the head, the oral care composition comprising an orally acceptable carrier and 1-20 wt. % of insoluble polymeric particulates having a mean particle size in the range of 3 to 10 μm, with at least 90 wt. % of the particles having a size below 15 μm, with respect to the weight of the cleaning composition.

An aspect of the disclosure is related to a cleaning composition that has relatively lower abrasivity. In some embodiments, a cleaning composition with relatively lower abrasivity is safe for daily use without damaging teeth or a dental appliance. In some embodiments, a cleaning composition has abrasivity that is lower than a commercially available or over the counter, conventional tooth paste. In some embodiments, the abrasivity can be determined by the Relative Dentin Abrasion (RDA) per ISO 11609. It measures how much dentin is removed by the test product using a standardized brushing procedure. An arbitrary RDA value of 100 is assigned to the Standard Reference material. Any toothpaste with RDA value below 250 can be recognized as safe for daily use by the American Dental Association. In some embodiments, a cleaning composition has abrasivity that is lower than a Standard Reference material that has an arbitrary RDA value of about 100. In some embodiments, a cleaning composition has an RDA value lower than about 250. In some embodiments, a cleaning composition has an RDA value lower than about 200. In some embodiments, a cleaning composition has an RDA value lower than about 175. In some embodiments, a cleaning composition has an RDA value lower than about 150. In some embodiments, a cleaning composition has an RDA value lower than about 125. In some embodiments, a cleaning composition has an RDA value lower than about 100. In some embodiments, a cleaning composition has an RDA value lower than about 90. In some embodiments, a cleaning composition has an RDA value lower than about 80. In some embodiments, a cleaning composition has an RDA value lower than about 70. In some embodiments, a cleaning composition has an RDA value lower than about 60. In some embodiments, a cleaning composition has an RDA value lower than about 50. In some embodiments, a cleaning composition has an RDA value lower than about 45. In some embodiments, a cleaning composition has an RDA value lower than about 40. In some embodiments, a cleaning composition has an RDA value lower than about 35. In some embodiments, a cleaning composition has an RDA value lower than about 30. In some embodiments, a cleaning composition has an RDA value lower than about 25. In some embodiments, a cleaning composition has an RDA value lower than about 24. In some embodiments, a cleaning composition has an RDA value from about 20 to about 30. In some embodiments, a cleaning composition has an RDA value from about 21 to about 28. In some embodiments, a cleaning composition has an RDA value from about 22 to about 26. In some embodiments, a cleaning composition has an RDA value from about 22.5 to about 25.5. In some embodiments, a cleaning composition has an RDA value from about 23 to about 25.

An aspect of the disclosure is related to that the composition is free of the aforementioned dental abrasive agents. Namely, the composition of the present invention has no abrasive material that is intentionally included. It has been unexpectedly found that the present composition can effectively clean teeth, remove stains and remove plaques, for example, by adding a hydrogel-based composition to achieve the cleaning effect. Moreover, due to the absence of dental abrasives, it is safe on cleaning dental appliances.

According to an aspect of the disclosure, the invented cleaning composition or oral care composition are in the form of dentifrices, such as toothpastes, tooth gels or tooth creams. Components of such toothpaste may include, with respect to the weight of the composition, a humectant (from about 10 wt. % to about 60 wt. %), a thickening agent (from about 0.1 wt. % to about 25 wt. %), a surfactant (from about 0.2 wt. % to about 10 wt. %), an essential oil component (from about 0.01 wt. % to about 3 wt. %) a coloring agent (from about 0.01 wt. % to about 0.5 wt. %) and water (from about 2 wt. % to about 40 wt. %). Such toothpaste may also include one or more of an additional active ingredient. Active ingredients include but not limited to, anticaries agents that provide a source of fluoride ion upon use, antimicrobial agents that reduce the formation of plaque on the surfaces, anti-inflammatory agents that prevent the inflammatory of gingiva. The preferred toothpaste compositions do not include abrasives that are solid particles having hardness higher than a surface of a dental appliance.

In preparing cleaning composition such as toothpaste, gel or cream, it is necessary to add thickening materials to provide a desirable consistency of the composition, to provide desirable release characteristics upon use, to provide shelf stability, and to provide stability of the composition, etc. Suitable thickening agents are carboxyvinyl polymers, water soluble polyvinylpyrrolidone (PVP), hydroxyethyl cellulose, carrageenan, laponite and water-soluble salts of cellulose ethers such as sodium carboxymethylcellulose, and sodium carboxymethyl hydroxyethyl cellulose. Natural gums such as xanthan gum, gum Arabic, gum karaya, and gum tragacanth can also be used. Hydrophilic organic particulates might be used as part of the thickening agent to further improve texture and handleability.

A suitable class of thickening or gelling agents includes a class of homopolymers of acrylic acid crosslinked with an alkyl ether of pentaerythritol or an alkyl ether of sucrose, or carbomers. Carbomers are commercially available as the Carbopol® series from Lubrizol. Particularly the Carbopols include Carbopol 940, 974, 980, and mixtures thereof.

Another suitable class of thickening or gelling agents includes a class of homopolymers and copolymers of acrylamidomethylpropanesulfonic acid and salts thereof may be crosslinked or uncrosslinked. In case of crosslinking, they contain structural units based on monomers having 2 or more olefinic double bonds. In the case of crosslinking, preferably from 0.1 mol % to 10 mol % of such structural units are present in the homo- or copolymers, based on the overall polymer. Preferable counterions are H⁺, Na⁺, and NH₄⁺. Suitable polymers include but not limited to Aristoflex® AVC (Ammonium Acryloyldimethyltaurate/VP copolymer), Aristoflex® AVS (Sodium AcryloyldimethyltaurateNP crosspolymer), and Aristoflex® HMB (Ammonium Acryloyldimethyl-taurate/Beheneth-25 methacrylate crosspolymer).

Thickening agents in an amount from about 0.1% to about 15%, or from about 0.2% to about 6%, in another embodiment from about 0.4% to about 2%, by weight of the total toothpaste, cream or gel composition, can be used.

According to an aspect of the disclosure, the cleaning composition 20 may include a mucosal adhesive. In some embodiments, a mucosal adhesive may enable a bioadhesion of the cleaning composition 20 to the oral cavity. Bioadhesion is a state in which two materials, at least one of which is biological in nature, are held together for a period of time by interfacial forces. Mucoadhesion is a type of bioadhesion in which one of the surfaces is mucus or a mucous membrane. As used herein, mucoadhesion also include adhesion in the oral cavity, such as adhesion to a surface involving saliva (e.g., a wet surface with saliva), biofilm, or mucosal area caused by bacteria. In a cleaning composition 20 as a cleaning agent for the oral cavity, the mucosal adhesive is to allow an adhesion time of the cleaning composition 20 to a dental appliance surface, which can be sufficient for the cleaning composition 20 to clean the dental appliance within the oral cavity. Bioadhesion such as adhesion to a surface in the oral cavity in presence of saliva is a complex phenomenon and multiple factors can influence the adhesion, including surrounding conditions in the oral cavity including conditions involving saliva, such as mucus properties, biofilm properties, bacterial mucus properties, saliva properties, dosage form characteristics, displacement forces such as a mechanical force from movements in the oral cavity, other substances present at the interface, other associated or correlated properties, and any combination thereof. Adhesion can involve consolidation of adhesion while adhesion is established, such as various physical and chemical interactions occurring between the mucosal adhesive and the surface in presence of saliva and biofilm to consolidate and strengthen the adhesion joint. The various physical and chemical interactions may include hydrogen bonding, macromolecular interpenetration, absorption of water, etc. Adhesion formed is temporary, and its duration can be determined by the strength of the adhesive forces and/or the mucus turnover. For example, dissociation of adhesion can occur at the interface between the dosage form and the mucus. For example, dissociation of adhesion can occur at the mucus layer (due to mucus turnover) or due to the whole system being overhydrated and washed out. Various materials or combinations of various materials can be used as a mucosal adhesive.

For example, Polyvinylpyrrolidone (PVP), also commonly called polyvidone or povidone, is a water-soluble polymer made from the monomer N-vinylpyrrolidone. PVP is available in a range of molecular weights and related viscosities, and can be selected according to the desired application properties. PVP has properties that can be controlled based on its molecular weight and polymerization method. For example, the rate of dissolution is inversely proportional to the molecular weight whereas the viscosity, complexing nature, and adhesive power are directly proportional to the molecular weight of PVP. Even the biological elimination of PVP upon parenteral administration is also inversely proportional to its molecular weight. Hence, a specific molecular weight PVP that is suitable for the desired application can be selected. By controlling the properties of the PVP, the adhesion properties such as adhesion duration can be controlled to achieve the cleaning ability of the cleaning composition 20.

For example, in some embodiments, sufficient duration of adhesion for a cleaning composition 20 can be about 10 seconds or longer until the dissociation of the cleaning composition adhesion. In some embodiments, sufficient duration of adhesion for a cleaning composition 20 can be about 15 seconds or longer until the dissociation of the cleaning composition adhesion. In some embodiments, sufficient duration of adhesion for a cleaning composition 20 can be about 20 seconds or longer until the dissociation of the cleaning composition adhesion. In some embodiments, sufficient duration of adhesion for a cleaning composition 20 can be about 30 seconds or longer until the dissociation of the cleaning composition adhesion. In some embodiments, sufficient duration of adhesion for a cleaning composition 20 can be about 10 seconds or longer until the dissociation of the cleaning composition adhesion. In some embodiments, sufficient duration of adhesion for a cleaning composition 20 can be about 15 seconds or longer until the dissociation of the cleaning composition adhesion. In some embodiments, sufficient duration of adhesion for a cleaning composition 20 can be about 20 seconds or longer until the dissociation of the cleaning composition adhesion. In some embodiments, sufficient duration of adhesion for a cleaning composition 20 can be about 25 seconds or longer until the dissociation of the cleaning composition adhesion. In some embodiments, sufficient duration of adhesion for a cleaning composition 20 can be about 30 seconds or longer until the dissociation of the cleaning composition adhesion. In some embodiments, sufficient duration of adhesion for a cleaning composition 20 can be about 40 seconds or longer until the dissociation of the cleaning composition adhesion. In some embodiments, sufficient duration of adhesion for a cleaning composition 20 can be about 50 seconds or longer until the dissociation of the cleaning composition adhesion. In some embodiments, sufficient duration of adhesion for a cleaning composition 20 can be about 1 minutes or longer until the dissociation of the cleaning composition adhesion. In some embodiments, sufficient duration of adhesion for a cleaning composition 20 can be about 2 minutes or longer until the dissociation of the cleaning composition adhesion. In some embodiments, sufficient duration of adhesion for a cleaning composition 20 can be about 3 minutes or longer until the dissociation of the cleaning composition adhesion. In some embodiments, sufficient duration of adhesion for a cleaning composition 20 can be about 4 minutes or longer until the dissociation of the cleaning composition adhesion. In some embodiments, sufficient duration of adhesion for a cleaning composition 20 can be about 5 minutes or longer until the dissociation of the cleaning composition adhesion.

A particulate rheology-modifying agent is included within the compositions of the invention to improve the rheological properties of the compositions. According to one embodiment, the rheology-modifying agent is included to offset the stringiness imparted by thickener and also to reduce or eliminate runniness. Rheology-modifying agents include insoluble hydrophilic polymers such as hydrophilic polyvinylpyrrolidone particulates commercially available as Kollidon and Luvicross, marketed by BASF, Mount Olive, N.J., USA, and POLYPLASDONE™ crospovidone polymer, marketed by Ashland Corporation, Wayne, N.J., USA. Other examples are modified celluloses such as cross-linked sodium carboxymethylcelluloses available as AC-DI-SOL®, PRIMELLO SE®, PHARMACEL XL®, EXPLOCEL®, and NYMCEL ZSX®, microcrystalline cellulose available as AVICEL®, PHARMACEL®, EMCOCELL®, VIVAPUR®, natural starches, such as maize starch, potato starch etc., modified starches such as carboxymethyl starches and sodium starch glycolate which are available as PRIMOJEL®, EXPLOTAB®, EXPLOSOL®, and starch derivatives such as amylose. In the present invention, each of them may be used solely or two or more thereof may be used jointly. The rheology-modifying agents may be included in a broad range from about 1% to about 25% by weight of the composition. A preferred mean particle size of the rheology-modifying agent is in the range of 3 to 10 μm. The mean particle size can be determined using a Malvern Mastersizer™. For the rheology-modifying agent, at least 90% of the particles by weight preferable have a size below 15 μm.

According to an aspect of the disclosure, an added abrasive agent can be eliminated and absence of the cleaning effect of an abrasive agent can be supplemented by a rheological effect, also defined herein as “lightweight abrasive” effect, or “rubbing” effect caused by shear force or viscous force.

According to an aspect of the disclosure, the cleaning composition 20 may include lightweight abrasive or rubbing agent different from abrasives, abrasive agent, dental abrasive, or dental abrasive agent. Various materials or combinations of various materials can be used as a lightweight abrasive or rubbing agent, which can provide a cleaning effect by shear force, viscous force or other form of force other than abrasion by hardness of a sold particle having hardness harder than a surface of a dental appliance, and which include various hydrogels and polymers. For example, cross-linked PVPs can be used to develop hydrogels due to its swelling properties in an aqueous environment.

According to an aspect of the disclosure, it was found that controlling the ratio between the amount of the water-soluble PVP and a surfactant unexpectedly increases the cleaning effect of the surfactant when compared to the cleaning composition without water-soluble PVP. It is considered that controlling the ratio of a surfactant and the water-soluble PVP increases the duration time of the surfactant on a surface being cleaned at a concentration effective enough to clean the surface. It is also considered that controlling the ratio of a surfactant and the water-soluble PVP can improve the spreadability of the cleaning composition, possibly due to the thickening effect of the water-soluble PVP or its contribution to the viscosity. In some embodiments, the increased cleaning effect was observed when the weight ratio between a cationic surfactant and water-soluble PVP was controlled to be from about 0.005 to about 2. In some embodiments, the increased cleaning effect was observed when the weight ratio between an amphiphilic surfactant and water-soluble PVP was controlled to be from about 0.005 to about 2. In some embodiments, the increased cleaning effect was observed when the weight ratio between a non-ionic surfactant and water-soluble PVP was controlled to be from about 0.005 to about 1. In some embodiments, the increased cleaning effect was observed when the weight ratio between total weight of all the surfactants in the cleaning composition and water-soluble PVP was controlled to be from about 0.01 to about 10. In some embodiments, the increased cleaning effect was observed when the weight ratio between a cationic surfactant and water-soluble PVP was controlled to be from about 0.01 to about 5. In some embodiments, the increased cleaning effect was observed when the weight ratio between an amphiphilic surfactant and water-soluble PVP was controlled to be from about 0.01 to about 2. In some embodiments, the increased cleaning effect was observed when the weight ratio between a non-ionic surfactant and water-soluble PVP was controlled to be from about 0.01 to about 1.

According to an aspect of the disclosure, an unexpected synergetic effect of combining two different forms of PVPs was found and applied to formulate a cleaning composition 20 without solid particles as an abrasive agent. According to an aspect of the disclosure, a water soluble PVP and a cross-linked PVP that is relatively less water-soluble or relatively not as water-soluble or relatively not water-soluble were combined together. It was unexpectedly found that, because both a water-soluble PVP and a cross-linked PVP share some molecular structural similarity, both PVPs were relatively more compatible to each other and relatively more miscible. Possibly due to the interaction of the cross-linked PVP that is not as water-soluble, the adhesive retention of the cleaning composition was unexpectedly and substantially increased when both a water-soluble PVP and a cross-linked PVP were included, compared to compositions containing a water-soluble PVP without a cross-linked PVP and compositions containing a cross-linked PVP without a water-soluble PVP. In some embodiments, adding a water-soluble PVP to a cross-linked PVP unexpectedly increased spreadability or dispersibility of the cross-linked PVP over a hard-to-reach surface and over a surface curvature of a dental appliance. In some embodiments, because of the compatibility between a water-soluble PVP and a cross-linked PVP, combining a water-soluble PVP and a cross-linked PVP unexpectedly allowed more diverse and more precise design of a property of the cleaning composition 20.

According to an aspect of the disclosure, controlling the ratio of a water-soluble PVP and a cross-linked PVP unexpectedly resulted in an improved effect of supplementing the solid particle-based adhesive agent. This is possibly because a controlled ratio of a water-soluble PVP and a cross-linked PVP unexpectedly increased shear force cleaning by increasing the spreadability over a surface while relatively maintaining viscous force by the cleaning composition 20 and possibly increased the adhesion of the cleaning composition 20. In particular, the unexpectedly increased cleaning effect of the cleaning composition 20 was observed when the ratio of a water-soluble PVP to a cross-linked PVP by weight (the weight of the water-soluble PVP/the weight of the cross-linked PVP) was from about 0.0025 to about 0.1. More substantial cleaning effect was observed when the ratio percent was from about 0.005 to about 0.05. More substantial cleaning effect was observed when the ratio percent was from about 0.01 to about 0.025, such as about 0.011, about 0.012, about 0.013, about 0.014, about 0.015, about 0.016, about 0.017, about 0.018, about 0.019, about 0.020, about 0.021, about 0.022, about 0.023, and about 0.024.

Humectant and water are used in the composition. The humectant serves to keep a cleaning composition or an oral care composition from hardening upon exposure to air, to give compositions a moist feel to the mouth, and for particular humectants, to impart desirable sweetness of flavor to the compositions. The humectant, on pure humectant basis, can comprise from about 0% to about 70%, and in another embodiment from about 20% to about 45%, by weight of the compositions herein. Suitable humectants for use in compositions of the subject invention include but not limited to edible polyhydric alcohols such as glycerin, sorbitol, xylitol, butylene glycol, polyethylene glycol, and propylene glycol, especially glycerin, sorbitol and xylitol, or any combination thereof. The compositions can contain 5% to 45% of water, more preferable 20% to 35% of water.

Sweetening agents which can be used include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, stevia and cyclamate salts, especially sodium saccharin, stevia, and the mixture thereof. A composition can contain from about 0.01% to about 30% of these agents, in another embodiment from about 0.1% to about 10% by weight of the composition.

According to an aspect of the disclosure, the oral care composition includes one or more surfactants. The surfactants enhance stability of the composition, help clean oral cavity surface through detergency, and provide foam upon mechanical motion, e.g., during brushing with an oral care composition of the disclosure. Acceptable surfactants for an oral cavity, such as anionic, nonionic, cationic, and amphoteric surfactants, can be used. A combination of surfactants may also be used. Suitable anionic surfactants include but not limited to water-soluble salts of C₈₋₁₂ alkyl sulfates, sulfonated monoglycerides of C₈₋₂₀ fatty acids, sarcosinates, taurates and the like. Examples of these includes sodium lauryl sulfate, sodium cocoyl monoglyceride sulfonate, sodium lauroyl sarcosinate, and disodium laureth sulfosuccinate. Suitable nonionic surfactants include without limitation to poloxamers, polyoxyethylene sorbitan esters, fatty alcohol ethxylates, alkylphenol ethoxylates, tertiary amino oxides, tertiary phosphine oxides dialkyl sulfoxides and the like. Suitable amphoteric surfactants include but not limited to derivatives of C₈₋₂₀ aliphatic secondary and tertiary amines having an anionic group such as carboxylate, sulfate, sulfonate, phosphate or phosphonate. A suitable example is cocamidopropyl betaine. Nonionic surfactants suitable for use in the composition of the present invention are generally produced by the condensation of alkylene oxide group with an organic hydrophobic compound which may be aliphatic or alkylaromatic in nature, for example polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixture thereof. In some embodiments, the nonionic surfactant comprises a block copolymer based on ethylene oxide and propylene oxide, such as poloxamer, also known by the trade name Pluronic®. In some embodiments, the nonionic surfactant is poloxamer 407, also known as Pluronic® F127 from BASF.

Any suitable flavoring agent may also be incorporated in the composition. Examples of suitable flavoring agents include flavoring essential oils, such as spearmint oil, peppermint oil, wintergreen oil, sassafras oil, sage oil, eucalyptus oil, marjoram oil, orange oil and methyl salicylate. Flavoring agents are included in the oral care composition in an amount of 0.01% to 3% by weight of the composition.

Pigments, dyes, colorants and lakes may also be added to modify the appearance of a cleaning composition or an oral care composition to render the product more acceptable to the consumer. Orally acceptable water-soluble dyes are preferred. Examples include but not limited to FD&C Green #1, FD&C Blue #1, FD&C Blue #2, FD&C Yellow #5, and combination thereof. Appropriate colorant levels are selected for the particular impact that is desirable to the consumer. The levels of dye and colorant may be in the range of about 0.001% to about 5%, in one embodiment from about 0.01% to about 0.5% by total weight of the oral care composition.

Oral care active agents which provide the intended therapeutic oral care benefit may also be added in the present invented composition. Oral care active agents include but no limited to anticalculus agents, antimicrobial agents, anti-plaque agents, anti-inflammatory agents, fluoride ion sources, whitening agents, antioxidants, and mixtures thereof.

Compositions of the present invention may also comprise an anticalculus agent from about 0.05% to about 50% by weight of the oral care composition, in another embodiment is from about 0.05% to about 25%, and in another embodiment is from about 0.1% to about 15%. The anticalculus agent may be selected from the group consisting of polyphosphates (including pyrophosphates) and salts, polyvinyl phosphates and salts, polyolefin phosphates and salts, polypeptides and mixtures. In one embodiment the pyrophosphate salt is selected from the group consisting of trisodium pyrophosphate, disodium dihydrogen pyrophosphate, dipotassium pyrophosphate, tetrasodium pyrophosphate, tetrapotassium pyrophosphate and mixture. Peptides include polyaspartic and its salts, polyglutamic acids and its salts may also be used.

Antimicrobial and antiplaque agents may be included in the compositions for the present invention. Such agents may include, but are not limited to chlorhexidine (CHX), benzalkonium chloride, cetylpyridinium chloride (CPC), iodine, sulfonamides, thymol, zinc or stannous ion agents, antibiotics such as augmentin, amoxicillin, tetracycline, doxycycline, minocycline, metronidazole, neomycin, kanamycin, cetylpyridinium chloride and clindamycin, methyl salicylate, hydrogen peroxide and mixtures of all of the above. Antimicrobial and antiplaque agents may be present from about 0.001% to about 20% by weight of the composition.

Anti-inflammatory agents can also be present in a cleaning composition or an oral care composition of the present invention. Such agents may include, but are not limited to, non-steroidal anti-inflammatory (NSAID) agents of oxicams, salicylates, propionic acid, acetic acids and fenamates. Such NSAIDs include but are not limited to ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone and acetaminophen.

Whitening agents can also be included in a cleaning composition or an oral care composition of the present invention. Suitable whitening agents are selected from the group consisting of alkali metal and alkaline earth metal peroxides, metal chlorites, perborates inclusive of mono and tetrahydrates, perphosphates, percarbonates, peroxyacids, alkali metal and persulfates, such as ammonium, potassium, sodium and lithium persulfates, and combinations thereof. Suitable peroxide compounds include hydrogen peroxide, urea peroxide, calcium peroxide, carbamide peroxide, magnesium peroxide, zinc peroxide, strontium peroxide and mixtures. In one embodiment the peroxide compound is hydrogen peroxide. The level of the whitening agent in the oral care composition is dependent on the available active oxygen that is capable of bleach the stain. In one embodiment, the whitening agents may be present at level from about 0.01% to about 20%, in another embodiment from about 0.1% to about 10%, and in another embodiment from about 3% to about 6%, by weight of the composition.

A fluoride ion source may be included in the oral care composition to provide an anticaries effect. Among these materials are inorganic fluoride salts, such as soluble alkali metal fluoride salts. Examples are sodium fluoride, potassium fluoride, sodium monofluorophosphate and sodium hexafluorosilicate. Alkali metal fluorides, such as sodium fluoride, sodium monofluorophosphate, sodium hexafluorosilicate and mixture are preferred.

A cleaning composition or an oral care composition optionally contain one or more other non-active ingredients. Non-limiting examples include pH modifying agents, foam modulators, and chelating agents. Toothpastes, tooth cream, tooth gels and other dentifrice compositions are formulated with these and optionally other additives according to known principles.

In one preferred aspect, the aforementioned cleaning composition, whether being a toothpaste, tooth cream or tooth gel, is positioned on a cleaning device 100 such as an oral care toothbrush. For example, the cleaning composition such as dentifrice can be positioned on the head of a cleaning device 100 such as an oral care toothbrush. This can be accomplished by positioning the cleaning composition such as dentifrice within or between the cleaning elements of a cleaning device 100 such as an oral care toothbrush. In other embodiment, this can be accomplished by coating, impregnating or otherwise incorporating or fixing the cleaning composition such as a dentifrice to the cleaning elements of a cleaning device 100 such as an oral care toothbrush. These concepts are described with reference to the drawings (FIGS. 1 and 2). When applied to such a tooth brushing device, the amount of the cleaning composition such as a dentifrice typically ranges 45 mg to 80 mg, preferably 60 mg to 70 mg, and most preferably about 70 mg of dentifrice.

In this disclosure, it was unexpectedly found that controlling the viscosity by formulating the cleaning composition in fact increase the cleaning effect of the cleaning composition without including abrasive agent that are commonly included in tooth pastes. It is considered that controlling the viscosity can increase a shear force applied to the surface being cleaned and also increase residual duration time of the cleaning composition on the surface. In some embodiments, the cleaning composition can have a relatively lower viscosity when compared to a commercially marketed tooth paste as a consumer product or as an over-the-counter product. In some embodiments, the cleaning composition can have a viscosity from about 500 cp to about 10,000 cp. In some embodiments, the cleaning composition can have a viscosity from about 600 cp to about 9,000 cp. In some embodiments, the cleaning composition can have a viscosity from about 700 cp to about 8,000 cp. In some embodiments, the cleaning composition can have a viscosity from about 800 cp to about 7,000 cp. In some embodiments, the cleaning composition can have a viscosity from about 900 cp to about 6,000 cp. In some embodiments, the cleaning composition can have a viscosity from about 1,000 cp to about 5,000 cp. In some embodiments, the cleaning composition can have a viscosity from about 1,300 cp to about 4,000 cp. In some embodiments, the cleaning composition can have a viscosity from about 1,500 cp to about 3,000 cp. In some embodiments, the cleaning composition can have a viscosity from about 1,800 cp to about 2,500 cp. In some embodiments, the cleaning composition can have a viscosity from about 1,900 cp to about 2,200 cp.

In some embodiments, the cleaning composition exhibit a level of the cleaning ability that is sufficient to clean the oral cavity or the dental appliance in the oral cavity.

In some embodiments, the Pellicle Cleaning Ratio (PCR) of the cleaning composition can be about 5% of the Standard Reference Material (SRM). In some embodiments, the Pellicle Cleaning Ratio (PCR) of the cleaning composition can be about 7% of the SRM. In some embodiments, the Pellicle Cleaning Ratio (PCR) of the cleaning composition can be about 10% of the SRM. In some embodiments, the Pellicle Cleaning Ratio (PCR) of the cleaning composition can be about 15% of the SRM. In some embodiments, the cleaning ability of the cleaning composition can be at least about 10% of a tooth paste having a PCR value of about 25%. In some embodiments, the cleaning ability of the cleaning composition can be at least about 10% of a tooth paste having a PCR value of about 25%. In some embodiments, the cleaning ability of the cleaning composition can be at least about 10% of a tooth paste having a PCR value of about 25%.

In some embodiments, the cleaning ability of the cleaning composition can be at least about 5% of a tooth paste having a PCR value of about 25%. In some embodiments, the cleaning ability of the cleaning composition can be at least about 7% of a tooth paste having a PCR value of about 25%. In some embodiments, the cleaning ability of the cleaning composition can be at least about 10% of a tooth paste having a PCR value of about 25%. In some embodiments, the cleaning ability of the cleaning composition can be at least about 15% of a tooth paste having a PCR value of about 25%. In some embodiments, the cleaning ability of the cleaning composition can be at least about 20% of a tooth paste having a PCR value of about 25%. In some embodiments, the cleaning ability of the cleaning composition can be at least about 30% of a tooth paste having a PCR value of about 25%. In some embodiments, the cleaning ability of the cleaning composition can be at least about 33% of a tooth paste having a PCR value of about 25%. In some embodiments, the cleaning ability of the cleaning composition can be at least about 35% of a tooth paste having a PCR value of about 25%. In some embodiments, the cleaning ability of the cleaning composition can be at least about 38% of a tooth paste having a PCR value of about 25%. In some embodiments, the cleaning ability of the cleaning composition can be at least about 40% of a tooth paste having a PCR value of about 25%. In some embodiments, the cleaning ability of the cleaning composition can be at least about 45% of a tooth paste having a PCR value of about 25%. In some embodiments, the cleaning ability of the cleaning composition can be at least about 50% of a tooth paste having a PCR value of about 25%.

### EXPERIMENTAL EXAMPLES

The following examples further describe and demonstrate the preferred embodiments within the scope of the present invention. The examples are given solely for the purpose of illustration, and are not be construed as limitations of the present invention since many variations thereof as possible without departing from its spirit and scope.

### Example 1: Cleaning Compositions

Cleaning compositions or an oral care composition were prepared as follows.

First, the salts were dissolved in water in a ROSS multi-shaft mixer at room temperature. Sorbitol, xylitol and glycerin were added and mixed by a disperser at 500 to 1000 rpm rotation speed. Thickeners, thickening modifiers, and other agents as present in the composition Tables 1 and 2 were added to the mixture and homogenized by a rotor stator at 2000 rpm rotation speed. After homogenization and de-aeration, all remaining components, including dyes, flavors and surfactants, were added and mixed by the disperser at a rotation rate of 500 rpm under vacuum. The obtained product is transferred into a compartment of the toothbrush illustrated in FIGS. 1 and 2.

TABLE 1
Compositions for Examples 1-3

| Component | Ex. 1 (wt. %) | Ex. 2 (wt. %) | Ex. 3 (wt. %) |
|---|---|---|---|
| Sodium saccharin | 0.1 | 0.15 | 0.1 |
| Sodium benzoate | 0.2 | 0.2 | 0.2 |
| Disodium EDTA | 0.5 | 0.5 | 0.5 |
| Dipotassium | 0.3 | 0.3 | 0.3 |
| phosphate | | | |
| Glycerin | 36 | 57 | 42 |
| Sorbitol | 10 | 10 | 10 |
| Xylitol | 5 | 5 | 5 |
| Xanthan gum | 0.5 | | 0.5 |
| Hydroxyethyl cellulose | 0.4 | 0.3 | |
| ARISTOFLEX ™ | | 0.5 | 0.5 |
| AVC | | | |
| Water-Insoluble | | | |
| Cross-linked | 10 | 13 | 12 |
| Polyvinylpyrrolidone | | | |
| Flavors | 0.3 | 0.6 | 0.3 |
| Dyes | 0.0005 | 0.0005 | 0.0005 |
| Sodium lauryl sulfate | 0.1 | 0.5 | 0.5 |
| Poloxamer | 0.5 | 0.5 | 0.5 |
| Cocamidopropyl | 0.5 | 1.0 | 1.0 |
| betaine | | | |
| Deionized water | QS to | QS to | QS to |
| | 100 wt. % | 100 wt. % | 100 wt. % |

According to an aspect of the disclosure, various cleaning compositions 20 were formulated. Various cleaning compositions 20 included any of the listed agent in Table 2, in various amounts, for example, any amounts from the weight percent (wt. %) ranges, with respect to the weight of the cleaning composition 20. In compositions including combinations of the water-soluble PVP and the cross-linked PVP, unexpected properties of increased cleaning effect were observed.

TABLE 2
Compositions including water-soluble PVP and cross-linked PVP

| Ingredient | CAS # | Function | wt. % | wt. % range |
|---|---|---|---|---|
| DI water | 7732-18-5 | solvent | 25.699% | 20-30% |
| Glycerin | 56-81-5 | humectant, solvent, | 39.000% | 30-50% |
| | | sweetener | | |
| Sorbitol | 50-70-4 | sweetener (sugar-less) | 1.000% | 0-15% |
| xylitol | 87-99-0 | sweetener (sugar-free) | 15.000% | 0-20% |
| Sodium saccharin | 6155-57-3 | sweetener (artificial) | 0.200% | 0-0.5% |
| Xanthan gum or | | thickener | 1.000% | 0.25-1.5% |
| hydroxyethyl | | | | |
| cellulose | | | | |
| Polyvinylpyrrolidone | 9003-39-8 | favor carrier, mucosal | 0.200% | 0-0.3% |
| (PVP) K30, | | adhesive | | |
| as water soluble | | | | |
| polyvinylpyrrolidone | | | | |
| PVP, Kollidon CL-M | 9003-39-8 | Lightweight abrasive | 15.000% | 3-20% |
| as crosslinked | | or rubbing agent, stain | | |
| polyvinylpyrrolidone, | | remover | | |
| water insoluble | | | | |
| Sodium lauryl sulfate | 151-21-3 | cationic surfactant | 0.100% | 0.01-2.0% |
| Chembetaine C | 61789-40-0 | amphiphilic surfactant | 1.000% | 0.5-2.0% |
| surfactant | | | | |
| Poloxamer 407 | 9003-11-6 | non-ionic surfactant | 0.500% | 0.5-1.0% |
| Na2EDTA | 6381-92-6 | calcium chelator, | 0.500% | 0-2.0% |
| | | plaque disruptor | | |
| K2HPO4 | 7758-11-4 | pH adjuster/buffer | 0.300% | 0.1-2.0% |
| Sodium benzoate | 532-32-1 | Preservative | 0.100% | 0.01-0.2% |
| Spearmint oil | 8008-79-5 | sweet mint favor | 0.150% | 0.01-0.5% |
| cornmint oil (wild | 68918-18-0 | mint favor | 0.000% | 0-0.5% |
| mint oil) | | | | |
| Benzoic acid | 65-85-0 | Preservative | 0.200% | 0-0.5% |
| Menthol | 2216-51-5 | cool mint favor | 0.050% | <0.01 |
| FD&C blue #1 | 3844-45-9 | color | 0.001% | <0.01 |
| Cetylpyridinium | 6004-24-6 | antimicrobial agent | 0 | 0-0.1% |
| chloride | | | | |
| methyl salicylate | 119-36-8 | antimicrobial agent | 0 | 0-0.1% |
| eucalyptol | 470-82-6 | anti-inflammatory | 0 | 0-0.1% |
| | | agent | | |
| thymol | 89-83-8 | antimicrobial agent | 0 | 0-0.1% |
| Chlorhexidine | 55-56-1 | antimicrobial agent | 0 | 0-1.0% |
| Zinc Lactate | 16039-53-5 | antimicrobial agent | 0 | 0-0.5% |
| Zinc citrate | 5990-32-9 | antiplaque and | 0 | 0-0.5% |
| | | antigingivitis agent | | |

The viscosity was controlled by different compositions to be within a range of about 1,000 cP to about 8,000 cP. One of the example viscosities was 2,000 cP.

The weight ratio between various surfactants and the insoluble PVP were controlled to meet at least one of the conditions below in Table 3:

TABLE 3

| Description | Range | Ratio range |
|---|---|---|
| Cationic surfactant (sodium | (0.1%-2%) vs (1%-20%) | 0.005-2 |
| lauryl sulfate) vs PVP | | |
| Amphiphilic surfactant | (0.1%-2%) vs (1%-20%) | 0.005-2 |
| (cocamidopropyl betaine) | | |
| vs PVP | | |
| Non-ionic surfactant | (0.1%-1%) vs (1%-20%) | 0.005-1 |
| (Poloxamer) vs PVP | | |
| Total surfactants vs PVP | (0.2%-10%) vs (1%-20%) | 0.01-10 |

### Example 2: Cleaning Ability

The cleaning ability of the compositions as the Pellicle Cleaning Ratio (PCR) were tested based on the cleaning ability of the cleaning composition to remove stained pellicle.

This laboratory test was developed in order to assess the ability of dentifrices to remove stained pellicle, i.e., to determine the cleaning ability of complete dentifrice formulations. A previous study (J. Dent. Res., 61:1236, 1982) has indicated that the results of this test with dentifrice slurries compare favorably with those obtained in controlled clinical trials. Thus, the results of this test using dentifrice slurries may be considered to predict clinical findings with a reasonable degree of confidence.

The compositions were compared to Reference silica slurry (Lot: 190061535, Evonik Resource Efficiency GmbH, Germany) (“Standard Reference Material”)

Specimen Preparation

Bovine, permanent, central incisors were cut to obtain labial enamel specimens approximately 10×10 mm. The enamel specimens were embedded in an autopolymerizing methacrylate resin so that only the enamel surfaces were exposed. The enamel surfaces were then smoothed and polished on a lapidary wheel and lightly etched to expedite stain accumulation and adherence. They were placed on a rotating rod (˜37° C. incubator), which alternately exposed them to air and to a solution consisting of PGY broth, tea, coffee, mucin, FeCl₃, and Micrococcus luteus.

The staining broth was changed, and specimens were rinsed daily until a uniform stain had accumulated. After approximately seven days, a darkly stained pellicle film was apparent on the enamel surfaces. Specimens were rinsed, allowed to air dry, and refrigerated until used. All products were tested using specimens prepared at the same time.

Scoring and Set-Up

The amount of in vitro stain was graded photometrically using only the L value of the L*a*b* scale using a spectrophotometer (Minolta CM2600d). The area of the specimens scored was a ¼-inch diameter circle in the center of the 10×10 mm enamel. Specimens with scores between 30 and 42 (30 being more darkly stained) were used. On the basis of these scores, the specimens were divided into groups of 16 specimens each, with each group having approximately the same average baseline score.

Procedure

The specimens were mounted on a mechanical V-8 cross-brushing machine equipped with soft nylonfilament (Oral-B 40) toothbrushes. Tension on the enamel surface will be adjusted to 150 g. The dentifrices were tested as slurries prepared by mixing 25 grams of dentifrice with 40 ml of deionized water. The Standard Reference Material was tested as slurry by mixing 10 g of reference silica with 50 ml 0.5% CMC solution. The specimens were brushed for 800 strokes (4½ minutes).

To minimize mechanical variables, two specimens per group were brushed on each of the eight brushing heads. Different test products were used on each run, with one tube of slurry made up for each product. Fresh slurry was made after being used to brush four specimens. Following brushing, specimens were rinsed, blotted dry, and scored again for stain, as previously described.

Calculations

The mean decrement between the pre- and post-brushing stain scores was determined for the Standard Reference Material group and assigned a pellicle cleaning ratio (PCR) value of 100. A constant value was calculated by dividing the mean decrement (corrected for the use of silica instead of Ca₂P₂O₇ as reference 3) of the Standard Reference Material into 100. The individual PCR value for each specimen was calculated by multiplying its individual decrement by the calculated constant. The mean, standard deviation, and SEM (standard error of the mean) for each test group (N=16) were then calculated using the individual PCR values. The larger the PCR value, the greater the amount of stained pellicle removed from the enamel surface in this test.

Data was analyzed using a t-test model [IBM SPSS Statistics Software]. All analyses were done with the significance level set at 0.05.

The results are summarized in the following Table 3, where PCR means (±SEM) are listed in descending order (higher mean PCR values represent higher efficacy on stained pellicle removal). The pre- and post-brushing scores and calculations are provided in the attached tables.

TABLE 3

| Cleaning Composition | Sample Size | Pellicle Cleaning Ratio |
|---|---|---|
| Standard Reference Material | 15 | 100.00 ± 2.32 |
| Cleaning Compositions in this | 15 | 9.84 ± 1.25 |
| disclosure | | |

It was found BlanX Non-abrasive Whitening toothpaste has a PCR value of 25 (https://www.researchgate.net/publication/49807515_Abrasion_polishing_and_stain_removal_characteristics_of_various_commercial_dentifrices_in_vitro). Although the cleaning compositions in this example was relatively less effective in stained pellicle removal than that of BlankX, it exhibited at least about 40% or more cleaning ability of BlankX. The cleaning composition without added solid particle abrasives also helps preventing damages on overdentures and implants as it is abrasive free and whitening free. Such a less abrasive effect was example in the next Example 3

In compositions including combinations of the water-soluble PVP and the cross-linked PVP, unexpected properties of increased cleaning effect were observed.

### Example 3: Relative Dentin Abrasion Level of the Cleaning Compositions

The cleaning ability of the compositions were tested based on the cleaning ability of the cleaning composition to remove stained pellicle.

The purpose of this study was to determine the relative abrasion level of the cleaning compositions.

The abrasivity was determined by the Relative Dentin Abrasion (RDA) per ISO 11609. It measures how much dentin is removed by the test product using a standardized brushing procedure. The procedure used in this study was the Hefferren abrasivity test recommended by the ADA and ISO 11609 for determination of dentifrice abrasiveness in dentin. The abrasivity limit specified by the ISO 11609 at 2.5× that of the Standard Reference Material (ISO Reference Silica) may be considered in the interpretation of the results of this test. Therefore, since the current protocol has assigned an arbitrary value of 100 to the Standard Reference Material, the RDA abrasivity limit is 250. Toothpastes with RDA value below 250 is recognized as safe for daily use by the American Dental Association.

Specimen Preparation

Eight (8) human dentin specimens were subjected to neutron bombardments resulting in the formation of radioactive phosphorus (32P) within the specimens under the controlled conditions outlined by the ADA. The specimens were mounted in methyl methacrylate so they fit in a V-8 cross-brushing machine. The specimens were brushed for a 1500 stroke, precondition run using slurry consisting of 10 g specified by the ADA with a brush tension of 150 g.

Procedure

Following the precondition run, the test was performed using the above parameters (150 g and 1500 strokes) in a “sandwich design.” Before and after brushing with the test product (25 g product/40 ml water) each tooth set was brushed with the Standard Reference Material (10 g of ISO Reference Silica/50 ml 0.5% CMC). The procedure was repeated additional times so that each product was assayed on each tooth set. The treatment design was the modified Latin Square design so that no treatment followed another treatment consistently.

Calculations

One ml samples were taken, each weighed (˜1 g), and added to 5 ml of “Ultima Gold′ scintillation cocktail. The samples were mixed well and immediately put on a liquid scintillation counter for radiation detection. Following counting, the net counts per minute (CPM) values were divided by the weight of the sample to calculate a net CPM/gram of slurry. The net CPM/g of the pre and post Standard Reference Material were multiplied by a correction factor 2 and then averaged to use in the calculation of RDA (relative dentin abrasion) for the test material. The Standard Reference Material was assigned a value of 100 and its ratio to the test material calculated.

The results are shown on the following table, where RDA mean values (±Standard Error of the Mean) are listed. Additionally, all raw data (individual specimen RDA values), the mean, the standard deviation, and standard error of the mean are reported on the attached table.

| Cleaning Composition | Sample Size | Pellicle Cleaning Ratio |
|---|---|---|
| Cleaning Compositions in this | 8 | 23.66 ± 1.10 |
| disclosure | | |

An arbitrary RDA value of 100 is assigned to the Standard Reference material. Any toothpaste with RDA value below 250 is recognized as safe for daily use by the American Dental Association. The RDA mean values (±Standard Error of the Mean) of the claimed composition (example 1) is 23.66±1.10. This value is below 2.5 times that of the reference material. Therefore, the composition is safe for daily use without damaging teeth. The test report is attached for reference.

Of note, example embodiments of the disclosure described above do not limit the scope of the invention since these embodiments are merely examples of the embodiments of the invention. Any equivalent embodiments are intended to be within the scope of this invention. Indeed, various modifications of the disclosure, in addition to those shown and described herein, such as alternative useful combinations of the elements described, may become apparent to those skilled in the art from the description. Such modifications and embodiments are also intended to fall within the scope of the appended claims.

What is claimed is:

1. A device to apply a cleaning composition to a dental appliance disposed in an oral cavity, comprising:
a container containing a cleaning composition to clean a surface of a dental appliance in an oral cavity;
an applicator portion to apply the cleaning composition to the surface of the dental appliance in the oral cavity and a gap between the dental appliance and the oral cavity, the applicator having a portion to be deformed based on the gap width of the gap to be inserted into the gap to apply the cleaning composition in the gap; and
an extruder to extrude the cleaning composition in the container through an outlet to the applicator portion and the portion,
wherein the cleaning composition comprises at least one surfactant and water-soluble Polyvinylpyrrolidone (PVP) at a ratio between the at least one surfactant and the water-soluble PVP of from about 0.01 to about 10,
wherein the cleaning composition is essentially free of a dental abrasive agent, wherein the dental abrasive agent is a solid particle having a hardness harder than the hardness of the surface of the dental appliance,
wherein the cleaning composition exhibits a viscosity of from about 500 cp to about 10,000 cp, and
wherein the cleaning composition exhibits about 10% of the cleaning ability of a tooth paste having a pellicle cleaning rate (PCR) value of about 25%.

2. The device of claim 1, wherein the portion is to be inserted into the gap by 3 millimeter (mm) or more.

3. The device of claim 1, wherein the gap width of the gap is about 0.3 mm.

4. The device of claim 1, wherein the cleaning composition further comprises a cross-linked polyvinylpyrrolidone (PVP),
wherein the weight ratio of the water-soluble PVP to the cross-linked PVP is from about 0.01 and to about 0.025.

5. The device of claim 1, wherein the weight percent of the cross-linked polyvinylpyrrolidone is about 3 wt. % to about 20 wt. %, with respect to the weight of the cleaning composition.

6. The device of claim 1, wherein the applicator portion includes a dispersing portion to disperse the cleaning composition over the surface of the dental appliance, wherein the dispersing portion includes the portion to be to be deformed.

7. The device of claim 6, wherein the dispersing portion is a brush to disperse the cleaning composition.

8. The device of claim 1, further comprising a cleaner portion to clean the surface of the dental appliance, wherein the cleaner portion includes the portion to be to be deformed.

9. The device of claim 8, wherein the cleaner portion includes a brush tip having bristles.

10. The device of claim 9, wherein an average length of the bristles is about 4 mm.

11. The device of claim 1, further comprising a handle to move the extruder to extrude the cleaning composition.

12. A device to apply a cleaning composition to a dental appliance disposed in an oral cavity, comprising:
a container containing a cleaning composition to clean a surface of a dental appliance in an oral cavity;
an applicator portion to apply the cleaning composition to the surface of the dental appliance in the oral cavity, the applicator having a portion to be deformed based on the gap width of the gap to be inserted into the gap to apply the cleaning composition in the gap; and
an extruder to extrude the cleaning composition in the container through an outlet to the applicator portion and the portion,
wherein the cleaning composition comprises at least one surfactant and water-soluble Polyvinylpyrrolidone (PVP) at a ratio between the at least one surfactant and the water-soluble PVP of from about 0.01 to about 10,
wherein the cleaning composition is essentially free of diamond, quartz, silicon carbide, alumina, silica gels and precipitates, calcium carbonate, titanium dioxide, talc, charcoal, and hydrophobic resinous abrasive materials,
wherein the cleaning composition exhibits a viscosity of from about 500 cp to about 10,000 cp, and
wherein the cleaning composition exhibits about 10% of the cleaning ability of a tooth paste having a pellicle cleaning rate (PCR) value of about 25%.

13. The device of claim 12, wherein the portion is to be inserted into the gap by 3 mm or more.

14. The device of claim 12, wherein the portion is to be inserted into the gap width of the gap is about 0.3 mm.

15. The device of claim 12, wherein the cleaning composition further comprises a cross-linked polyvinylpyrrolidone (PVP),
wherein the weight ratio of the water-soluble PVP to the cross-linked PVP is from about 0.01 and to about 0.025.

16. The device of claim 12, wherein the weight percent of the cross-linked polyvinylpyrrolidone is about 3 wt. % to about 20 wt. %, with respect to the weight of the cleaning composition.

17. The device of claim 12, wherein the applicator portion includes a dispersing portion to disperse the cleaning composition over the surface of the dental appliance,
wherein the dispersing portion includes the portion to be to be deformed, and
wherein the dispersing portion includes a brush to disperse the cleaning composition.

18. The device of claim 12, further comprising a cleaner portion to clean the surface of the dental appliance,
wherein the cleaner portion includes the portion to be to be deformed,
wherein the cleaner portion includes a brush tip having bristles, and
wherein an average length of the bristles is about 4 mm.

19. A dental appliance cleaning composition to clean a surface of a dental appliance in an oral cavity, comprising:
a solvent;
a water-soluble polyvinylpyrrolidone (PVP); and
at least one surfactant,
wherein the cleaning composition comprises at least one surfactant and the water-soluble PVP at a ratio between the at least one surfactant and the water-soluble PVP of from about 0.01 to about 10,
wherein the cleaning composition is essentially free of a dental abrasive agent, wherein the dental abrasive agent is a solid particle having a hardness harder than the hardness of the surface of the dental appliance,
wherein the cleaning composition exhibits a viscosity of from about 500 cp to about 10,000 cp, and
wherein the cleaning composition exhibits about 10% of the cleaning ability of a tooth paste having a pellicle cleaning rate (PCR) value of about 25%.

20. The dental appliance cleaning composition of claim 19, wherein the cleaning composition further comprises a cross-linked polyvinylpyrrolidone (PVP),
wherein the weight ratio of the water-soluble PVP to the cross-linked PVP is from about 0.01 and to about 0.025.
